(19) United States
(12) Patent Application Publication
(10) Pub. No.: US 2023/0338283 A1
(43) Pub. Date: Oct. 26, 2023

(54) PHARMACEUTICAL FORMULATION

(71) Applicant: Agios Pharmaceuticals, Inc., Cambridge, MA (US)

(72) Inventors: Cheuk-Yui Leung, Acton, MA (US); Eric Simone, West Newbury, MA (US); Ophelia Qiping Yin, Westfield, NJ (US)

(21) Appl. No.: 18/028,058

(22) PCT Filed: Sep. 24, 2021
(86) PCT No.: PCT/US2021/051957

§ 371 Date: Mar. 23, 2023

Related U.S. Application Data
(60) Provisional application No. 63/238,483, filed on Aug. 30, 2021.
(60) Provisional application No. 63/107,196, filed on Oct. 29, 2020.
(60) Provisional application No. 63/083,834, filed on Sep. 25, 2020.

(51) Int. Cl. A61K 9/00; A61K 31/496; A61K 9/16; A61K 9/20

(52) U.S. Cl. CPC A61K 9/0056; A61K 31/496; A61K 9/1652; A61K 9/2054

(57) ABSTRACT

Disclosed is a pharmaceutical mini-tablet comprising mitapivat or a pharmaceutically acceptable salt thereof, and in a particular embodiment comprising a mitapivat sulfate salt, and one or more inactive ingredients selected from a filler, disintegrant, lubricant, binder, and diluent.

### REFERENCE TO RELATED APPLICATIONS

This application claims the benefit of the filing dates of U.S. Provisional Patent Application Nos. 63/083,834, filed on Sep. 25, 2020, 63/107,196, filed on Oct. 29, 2020, and 63/238,483, filed on Aug. 30, 2021, the entire contents of each of which are incorporated herein by reference.

### BACKGROUND

Pyruvate kinase deficiency (PKD) is a disease of the red blood cells caused by a deficiency of the pyruvate kinase R (PKR) enzyme due to recessive mutations of PKLR gene (Wijk et al. Human Mutation, 2008, 30 (3) 446-453). PKR activators can be beneficial to treat PKD, thalassemia (e.g., beta-thalessemia), abetalipoproteinemia or Bassen-Kornzweig syndrome, sickle cell disease, paroxysmal nocturnal hemoglobinuria, anemia (e.g., congenital anemias (e.g., enzymopathies), hemolytic anemia (e.g. hereditary and/or congenital hemolytic anemia, acquired hemolytic anemia, chronic hemolytic anemia caused by phosphoglycerate kinase deficiency, anemia of chronic diseases, non-spherocytic hemolytic anemia or hereditary spherocytosis). Treatment of PKD is supportive, including blood transfusions, splenectomy, chelation therapy to address iron overload, and/or interventions for other disease-related morbidity. Currently, however, there is no approved medicine that treats the underlying cause of PKD, and thus the etiology of life-long hemolytic anemia.

N-(4-(4-(cyclopropylmethyl)piperazine-1-carbonyl)phenyl)quinoline-8-sulfonamide, also known as mitapivat, is an allosteric activator of red cell isoform of pyruvate kinase R (PKR). See e.g., WO 2011/002817 and WO 2016/201227, the contents of which are incorporated herein by reference.

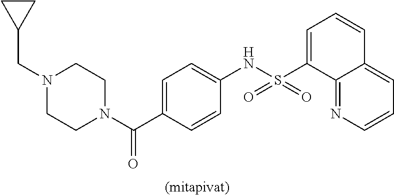 [SMILES: O=C(c1ccc(NS(=O)(=O)c2cccc3cccnc23)cc1)N1CCN(CC2CC2)CC1]

Mitapivat (whether administered as a free base or as a pharmaceutically acceptable salt) was developed to treat PKD and other blood disorders, and is currently being investigated in phase 2 and phase 3 clinical trials. See e.g., U.S. clinical trials identifier NCT02476916. Given its therapeutic benefits, there is a need to develop suitable formulations for the effective delivery of mitapivat and/or its pharmaceutical salts to a variety of different patients, including pediatric and elderly patients as well as patients who have difficulty swallowing traditional tablets.

### SUMMARY

A hemisulfate sesquihydrate salt of mitapivat, namely 1-(cyclopropylmethyl)-4-(4-(quinoline-8-sulfonamido)benzoyl)piperazin-1-ium hemisulfate sesquihydrate), referenced in one aspect herein as mitapivat hemisulfate sesquihydrate or the hemisulfate sesquihydrate, has been developed as 5 mg, 20 mg, 50 mg, and 100 mg strength tablets, in which the amount of mitapivat hemisulfate sesquihydrate present in the tablet corresponds to an amount that is equivalent to 5 mg, 20 mg, 50 mg, and 100 mg of mitapivat respectively. Given that there are populations of patients who may have difficulty swallowing traditional tablets and/or may require smaller doses of mitapivat, e.g., pediatric patients, elderly patients and patients with illnesses that effect swallowing or patients who have difficulty swallowing traditional tablets for other reasons, there is a need for smaller and more easily swallowed dosage forms that can be administered to these patients. In some embodiments, disclosed herein are mini tablets having, as its longest dimension or diameter, a length of about 5.0 mm to about 1.0 mm (e.g. about 2.5 mm to about 2.0 mm) with about 0.1 mg to about 1.5 mg strength of mitapivat or a pharmaceutically acceptable salt thereof in an amount that is equivalent to about 0.1 mg to about 5 mg of mitapivat) per minitablet to address this need. More specifically, the present application provides for a mini-tablet dosage form comprising about 0.5 mg to about 1.5 mg of mitapivat or an amount of a pharmaceutically acceptable salt of mitapivat that is equivalent to about 0.5 mg to about 1.5 mg of mitapivat.

It has also been found that mitapivat and its pharmaceutically acceptable salts such as mitapivat hemisulfate sesquihydrate tend to have aversive sensory attributes, including a bitter basic taste, green stemmy aroma and a tannin mouth feel. Flavor is critically important to the successful administration of oral medicines, particularly in pediatric patients. For the young pediatric population, it is common to dose a drug together with a food vehicle. It is also important that the food and dosage form mixture do not carry the aversive sensory attributes that affect the acceptability of the drug by the patient. It has now been found that out of the various taste masking food vehicles tested, chocolate pudding, strawberry jam and peanut butter, masked the bitter taste of tablets or minitablets comprising mitapivat or a pharmaceutically acceptable salt thereof with the most favorable profile, such that the bitter taste was not perceptible to patients (see Examples 1 and 2). In addition, strawberry yogurt and apple sauce showed an acceptable profile to mask the bitter taste of the disclosed tablets or mini-tablets comprising mitapivat or a pharmaceutically acceptable salt thereof.

Provided herein, therefore, are methods for treating conditions comprising administering to a pediatric subject in need thereof, a pharmaceutically effective amount of mitapivat or a pharmaceutically acceptable salt thereof together with food or water. In one aspect, provided are methods of treating conditions such as pyruvate kinase deficiency, sickle cell disease, and thalassemia in a pediatric subject using a therapeutically effective amount of mitapivat or a pharmaceutically acceptable salt thereof together with food or water. In some aspects, the pediatric subject is between about 1 year of age to about 12 years of age. In some aspects, the pediatric subject has a body weight of about 7 kg to less than about 40 kg. In some aspects the pediatric subject is between about 1 year of age to about 12 years of age and has a body weight of about 7 kg to less than about 40 kg. In some aspects the pediatric subject is between about 1 year of age to less than about 2 years of age. In other aspects the pediatric subject has a body weight of about 20 kg to less than about 40 kg. In other aspects the pediatric subject has a body weight of about 7 kg to less than about 20 kg. In some aspects, the subject is administered a pharmaceutically acceptable salt such as (1-(cyclopropylmethyl)-4-(4-(quinoline-8-sulfonamido)benzoyl)piperazin-1-ium hemisulfate sesquihydrate). In some aspects, the mitapivat or a pharmaceutically acceptable salt thereof is in the form of one or more granules, e.g., a pharmaceutical minitablet comprising about 0.1 mg to about 5 mg of mitapivat or a pharmaceutically acceptable salt thereof in an amount that is equivalent to about 0.1 mg to about 5 mg of mitapivat, microcrystalline cellulose, mannitol, croscarmellose sodium and sodium stearyl fumarate. In some aspects, the subject has PKD. In other aspects, the subject has thalassemia. In still other aspects, the subject has sickle cell disease.

One embodiment set forth herein is a pharmaceutical minitablet comprising an amount of mitapivat or a pharmaceutically acceptable salt thereof, such as mitapivat sulfate, and one or more inactive ingredients selected from a filler, a disintegrant, a lubricant, a binder, and a diluent. In some aspects the minitablet has a film coat. In one aspect, the minitablets are suitable for administration with a food vehicle that effectively masks the aversive sensory attributes of mitapivat or its pharmaceutically acceptable salts including mitapivat sulfate specifically.

Another embodiment set forth herein is a pharmaceutical unit dosage system comprising one or more of the mini-tablets disclosed herein. In one aspect, the unit dose is arranged to deliver mitapivat or a pharmaceutical acceptable salt thereof, in an amount of about 1 mg to about 200 mg. In some embodiments, the unit dose is arranged to deliver mitapivat or a pharmaceutical acceptable salt thereof, in an amount of about 0.25 mg, about 0.5 mg, about 1 mg, about 2 mg, about 3 mg, about 4 mg, about 5 mg, about 6 mg, about 7 mg, about 8 mg, about 9 mg, about 10 mg, about 16 mg, about 15 mg, about 30 mg, about 25 mg, about 50 mg, about 60 mg, about 70 mg, about 75 mg, about 80 mg, about 90 mg, or about 100 mg. In some embodiments, the unit dose is arranged to deliver mitapivat or a pharmaceutically acceptable salt thereof, in an amount of about 16 mg, about 30 mg, about 50 mg, or about 100 mg for a thalassemia subject once or twice daily. In some embodiments the unit dose is a single minitablet that is arranged to deliver mitapivat or a pharmaceutically acceptable salt thereof in an amount of 0.25 mg, about 0.5 mg, about 1 mg, about 2 mg, about 3 mg, about 4 mg, about 5 mg, about 6 mg, about 7 mg, about 8 mg, about 9 mg, about 10 mg. In other embodiments the unit dose is a single minitablet that is arranged to deliver mitapivat or a pharmaceutically acceptable salt thereof in an amount of 0.25 mg, about 0.5 mg, about 1 mg, about 2 mg. In some embodiments, the unit dose is arranged to deliver mitapivat or a pharmaceutical acceptable salt thereof, in an amount described herein to a subject with PKD. In still other embodiments, the unit dose is arranged to deliver mitapivat or a pharmaceutical acceptable salt thereof, in an amount described herein to a subject with sickle cell disease. In some embodiments, the unit dose is arranged to deliver mitapivat of about 16 mg to a thalassemia subject of about one year old to about two years old. In some embodiments, the unit dose is arranged to deliver mitapivat of about 30 mg to a thalassemia subject of about two years old to about six years old. In some embodiments, the unit dose is arranged to deliver mitapivat of about 50 mg to a thalassemia subject of about six years old to about twelve years old. In some embodiments, the unit dose is arranged to deliver mitapivat of about 100 mg to a thalassemia subject of about twelve years old to about eighteen years old. In some embodiments, the unit dose is arranged to deliver mitapivat of about 100 mg to a thalassemia subject of at least eighteen years old. In some embodiments, the unit dose is arranged to deliver mitapivat of about 16 mg, about 30 mg, about 50 mg, or about 100 mg for a thalassemia subject once daily. In some embodiments, the unit dose is arranged to deliver mitapivat of about 10 mg, about 15 mg, about 25 mg, about 50 mg for a PKD subject twice daily. In some embodiments, the unit dose is arranged to deliver mitapivat of about 10 mg to a PKD subject of about one year old to about two years old. In some embodiments, the unit dose is arranged to deliver mitapivat of about 15 mg to a PKD subject of about two years old to about six years old. In some embodiments, the unit dose is arranged to deliver mitapivat of about 25 mg to a PKD subject of about six years old to about twelve years old. In some embodiments, the unit dose is arranged to deliver mitapivat of about 50 mg to a PKD subject of about twelve years old to about eighteen years old. In some embodiments, the unit dose is arranged to deliver mitapivat of about 50 mg to a PKD subject of at least twelve years old. In some embodiments, the unit dose is arranged to deliver mitapivat of about 10 mg, about 15 mg, about 25 mg, about 50 mg for a PKD subject once daily. The pharmaceutical unit dosage system, for example, is a sachet or a stick pack, in which the disclosed mini tablets are packed.

The present disclosure further provides a method of treating anemia in a subject (e.g., a pediatric patient, an elderly patient or a patient who has difficulty swallowing) comprising administering to the subject a pharmaceutically effective amount of one or more mini-tablets disclosed herein. In certain embodiments, the anemia is a dyserythropoietic anemia such as congenital dyserythropoietic anemia type I, II, III, or IV.

The present disclosure further provides a method for treating hemolytic anemia (e.g., chronic hemolytic anemia caused by phosphoglycerate kinase deficiency, Blood Cells Mol Dis, 2011; 46(3):206) in a subject (e.g., a pediatric patient, an elderly patient or a patient who has difficulty swallowing) comprising administering to the subject a pharmaceutically effective amount of a mini-tablet disclosed herein. In certain embodiments, the hemolytic anemia is hereditary and/or congenital hemolytic anemia, acquired hemolytic anemia, or anemia as part of a multi-system disease. In certain embodiments, the hemolytic anemia is congenital anemia. In certain embodiments, the hemolytic anemia is hereditary (e.g. non-spherocytic hemolytic anemia or hereditary spherocytosis).

The present disclosure further provides a method for treating sickle cell disease (SCD) in a subject (e.g., a pediatric patient, an elderly patient or a patient who has difficulty swallowing) comprising administering to the subject a pharmaceutically effective amount of a mini-tablet disclosed herein. In some embodiments, the provided method reduces the vaso-occlusive crises (VOC) in the SCD subject. In some embodiments, the method further comprises administering hydroxyurea (HU) in the SCD subject.

The present disclosure further provides a method for treating thalassemia (e.g., alpha-thalassemia and/or beta-thalassemia), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), sickle cell disease, or anemia of chronic diseases in a subject (e.g., a pediatric patient, an elderly patient or a patient with a disease or condition that makes swallowing difficult) comprising administering to the subject (e.g., a pediatric patient, an elderly patient or a patient who has difficulty swallowing) a pharmaceutically effective amount of a mini-tablet disclosed herein. In one embodiment, the acquired hemolytic anemia comprises congenital anemias. In certain embodiments, the provided method is to treat thalassemia. In certain embodiments, the thalassemia is beta-thalassemia. In certain embodiments, the thalassemia is alpha-thalassemia.

The present disclosure further provides a method for treating pyruvate kinase deficiency (PKD) in a subject (e.g., a pediatric patient, an elderly patient or a patient who has difficulty swallowing), the method comprising administering to the subject a pharmaceutically effective amount of a mini-tablet disclosed herein. In this aspect of the invention, the PKD is a deficiency of PKR. The deficiency of PKR is associated with a pyruvate kinase R mutation.

Mitapivat is an activator of PKR having lower activities compared to the wild type, and therefore is useful for the methods of the present disclosure. In certain embodiments, the PKR is a wild type. In certain embodiments, the PKR is a mutant. Such mutations in PKR can affect enzyme activity (catalytic efficiency), regulatory properties (modulation by fructose bisphosphate (FBP)/ATP), and/or thermostability of the enzyme. Examples of such mutations are described in Valentini et al, JBC 2002. Some examples of the mutants that are activated by the compounds described herein include G332S, G364D, T384M, R479H, R479K, R486W, R532W, K410E, R510Q, and R490W.

In an embodiment, the disclosure provides a method for activating PKR in red blood cells in a subject (e.g., a pediatric patient, an elderly patient or a patient who has difficulty swallowing) with PKD comprising administering to the subject a pharmaceutically effective amount of a mini-tablet disclosed herein. In certain embodiments, the PKR is wild type PKR. In certain embodiments, the PKR is a mutant PKR.

In an embodiment, the mutant PKR is selected from G332S, G364D, T384M, K410E, R479H, R479K, R486W, R532W, R510Q, and R490W. In certain embodiments, the mutant PKR is selected from A468V, A495V, I90N, T408I, and Q421K, and R498H. In certain embodiments, the mutant PKR is R532W, K410E, or R510Q.

### BRIEF DESCRIPTION OF THE FIGURES

FIG. 1 is a table showing a proposed dose for mitapivat according to age and body weight for treating pyruvate kinase deficiency and thalassemia.

FIG. 2 is a graph showing bitterness intensity over time in minutes experienced by sensory panelists administered three whole 5 mg strength mitapivat tablets in nine different food dosing vehicles (Example 1).

FIG. 3 is a graph showing bitterness intensity over time in minutes experienced by sensory panelists administered three crushed 5 mg strength mitapivat tablets in different nine food dosing vehicles (Example 1).

FIG. 4 is a bar graph showing the mean time to “bitter breakthrough” in panelists administered mitapivat mini-tablets.

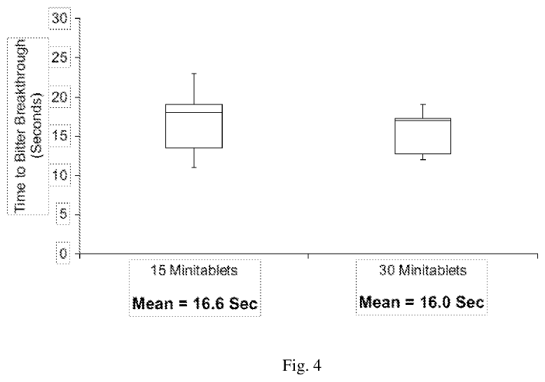

FIG. 5 is a graph showing the bitterness intensity over time in minutes experienced by sensory panelists administered fifteen or thirty 1.0 mg strength mitapivat mini-tablets in unsweetened apple sauce (Example 2).

FIG. 6 is a graph showing the bitterness intensity over time in minutes experienced by sensory panelists administered fifteen or thirty 1.0 mg strength mitapivat mini-tablets in sweetened apple sauce (Example 2).

FIG. 7 is a graph a graph showing the bitterness intensity over time in minutes experienced by sensory panelists administered fifteen or thirty 1.0 mg strength mitapivat mini-tablets in chocolate pudding (Example 2).

FIG. 8 is a graph a graph showing the bitterness intensity over time in minutes experienced by sensory panelists administered fifteen or thirty 1.0 mg strength mitapivat mini-tablets in strawberry yogurt (Example 2).

### DETAILED DESCRIPTION

In one aspect the application is directed to a mini-tablet comprising mitapivat or a pharmaceutically acceptable salt thereof including mitapivat sulfate and a pharmaceutical unit dosage form thereof. In one aspect this application provides a mini-tablet dosage form comprising an amount of mitapivat or a pharmaceutically acceptable salt thereof. In another aspect, the application provides mini-tablet dosage form comprising a mitapivat sulfate salt such as the hemisulfate sesquihydrate salt and one or more inactive ingredients selected from a filler, disintegrant, lubricant, binder, and diluent. In a specific aspect, the mini-tablet comprises a mitapivat sulfate salt and one or more inactive ingredients selected from microcrystalline cellulose, croscarmellose sodium, sodium stearyl fumarate, and mannitol. Optionally, the mini tablet further comprises a film coat. In one aspect, the mini-tablet is of about 0.1 mg to about 50.00 mg strength (i.e. equivalent to about 0.1 mg to about 50.00 mg of mitapivat or an amount of a pharmaceutically acceptable salt equivalent to about 0.1 mg to about 50.00 mg of mitapivat). In another aspect, the mini-tablet is of about 0.5 mg, about 1.0 mg, about 5.0 mg, or about 10.00 mg strength (i.e. equivalent to about 0.5 mg, about 1.0 mg, about 5.0 mg, or about 10.00 mg of mitapivat or an amount of a pharmaceutically acceptable salt equivalent to about 0.5 mg, about 1.0 mg, about 5.0 mg, or about 10.00 mg of mitapivat). In another aspect, the mini-tablet is of about 1.0 mg strength (i.e. equivalent to about 1 mg of mitapivat or an amount of a pharmaceutically acceptable salt equivalent to about 1 mg of mitapivat).

“Mini-tablets”, “Minitablets”, “Mini tablets”, “Minitabs,” “granules” and “pellets” are used interchangeably herein and mean one or more small particles suitable for oral administration comprising mitapivat or a pharmaceutically acceptable salt thereof with each mini-tablet having as its longest dimension or diameter, a length of less than about 10.0 mm. In one aspect, the disclosed mini-tablet has a shape that is cylinder-like, oval-like, cone-like, sphere-like, ellipsis-like, polygon-like or combinations thereof. In other embodiments the mini-tablet has, as its longest dimension or diameter, a length of about 10.0 mm to about 0.1 mm. Alternatively, the mini-tablet has, as its longest dimension or diameter, a length of about 5.0 mm to about 0.1 mm. In another alternative, the mini-tablet has, as its longest dimension or diameter, a length of about 2.5 mm to about 2.0 mm.

In one aspect the mini-tablets described herein are comprised of mitapivat. In one aspect the mini-tablets described herein are comprised of a pharmaceutically acceptable salt of mitapivat. In one aspect, the mini-tablets are comprised of a mitapivat sulfate salt. In one aspect, the mitapivat sulfate salt is a hydrate. In another aspect, the mitapivat sulfate hydrate salt is referred to as “1-(cyclopropylmethyl)-4-(4-(quinoline-8-sulfonamido)benzoyl)piperazin-1-ium hemisulfate sesquihydrate” which is represented by Formula A as shown below (also referred to as mitapivat hemisulfate sesquihydrate), or alternatively “1-(cyclopropylmethyl)-4-(4-(quinoline-8-sulfonamido)benzoyl)piperazin-1-ium sulfate trihydrate” which is represented by Formula B as shown below:

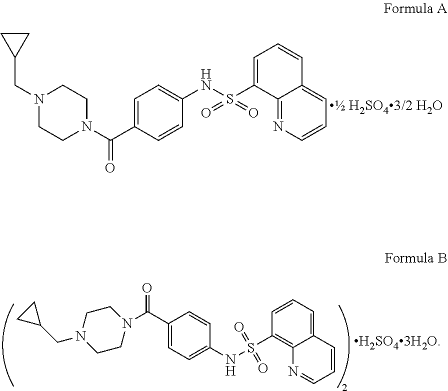

The mitapivat sulfate hydrate salt (e.g., as in Formula A or Formula B) can be crystalline, for example, Form A as disclosed in U.S. Publication No. 20200277279, the entire teachings of which are incorporated herein by reference. Form A is characterized by any one of the following x-ray powder diffraction patterns at 2Θ angles (±0.2°) using Cu Kα radiation: 9.9°, 15.8°, and 22.6′; 15.0°, 17.1°, 21.3°, and 21.9′; 9.9°, 15.0°, 15.8°, 17.1°, 21.3°, 21.9°, and 22.6′; 9.9°, 11.4°, 15.0°, 15.3°, 15.8°, 17.1°, 17.7°, 21.3°, 21.9°, 22.6°, and 23.5°; or 4.9°, 9.9°, 11.0°, 11.4°, 11.7°, 12.3°, 12.8°, 13.6°, 13.9°, 14.2°, 15.0°, 15.3°, 15.8°, 17.1°, 17.4°, 17.7°, 18.8°, 19.1°, 19.8°, 21.3°, 21.9°, 22.6°, 23.0°, 23.2°, 23.5°, 23.8°, 24.1°, 24.5°, 25.3°, 25.6°, 26.1°, 27.1°, 28.1°, and 29.8°. In some embodiments, Form A is characterized by x-ray powder diffraction peaks at 2Θ angles (±0.2°) 9.9°, 15.8°, and 22.6°. In certain embodiments, Form A is characterized by x-ray powder diffraction peaks at 2Θ angles (±0.2°) 9.9°, 15.8°, and 22.6° and at least one additional x-ray powder diffraction peak at 2Θ angles (±0.2°) selected from 15.0°, 17.1°, 21.3°, and 21.9°. In certain embodiments, Form A is characterized by x-ray powder diffraction peaks at 2Θ angles (±0.2°) 9.9°, 15.8°, and 22.6°; and at least two additional x-ray powder diffraction peaks at 2Θ angles (±0.2°) selected from 15.0°, 17.1°, 21.3°, and 21.9°. In yet another alternative, Form A is characterized by x-ray powder diffraction peaks at 2Θ angles (±0.2°) 9.9°, 15.8°, and 22.6°; and at least three additional x-ray powder diffraction peaks at 2Θ angles (±0.2°) selected from 15.0°, 17.1°, 21.3°, and 21.9°. In certain embodiments, Form A is characterized by x-ray powder diffraction peaks at 2Θ angles (±0.2°) 9.9°, 15.0°, 15.8°, 17.1°, 21.3°, 21.9°, and 22.6°. In certain embodiments, Form A is characterized by x-ray powder diffraction peaks at 2Θ angles (±0.2°) 9.9°, 11.4°, 15.0°, 15.3°, 15.8°, 17.1°, 17.7°, 21.3°, 21.9°, 22.6°, and 23.5°. In certain embodiments, Form A is characterized by x-ray powder diffraction peaks at 2Θ angles (±0.2°) 4.9°, 9.9°, 11.0°, 11.4°, 11.7°, 12.3°, 12.8°, 13.6°, 13.9°, 14.2°, 15.0°, 15.3°, 15.8°, 17.1°, 17.4°, 17.7°, 18.8°, 19.1°, 19.8°, 21.3°, 21.9°, 22.6°, 23.0°, 23.2°, 23.5°, 23.8°, 24.1°, 24.5°, 25.3°, 25.6°, 26.1°, 27.1°, 28.1°, and 29.8°. In yet another alternative, Form A is characterized by a differential scanning calorimetry (DSC) thermograph comprising endotherm peaks at about 159° C.±5° C. and 199° C.±5° C. In yet another alternative, crystalline Form A is characterized by a thermogravimetric analysis (TGA) thermogram comprising a weight loss of about 4.5±0.5% up to 180° C.±2° C. In some embodiments, the mitapivat sulfate hydrate salt is 1-(cyclopropylmethyl)-4-(4-(quinoline-8-sulfonamido)benzoyl)piperazin-1-ium hemisulfate sesquihydrate Form A.

As used herein, the “strength” of a minitablet refers to the corresponding amount of mitapivat or an amount of a pharmaceutically acceptable salt of mitapivat that is equivalent to the same amount of mitapivat (i.e., the free base) present in each minitablet. In some embodiments, the minitablet has the strength of about 0.1 mg to about 5.0 mg of mitapivat or an amount of a pharmaceutically acceptable salt of mitapivat that is equivalent to about 0.1 mg to about 5.0 mg of mitapivat.

In some embodiments, the minitablet comprises a mitapivat sulfate salt. In some embodiments, the mitapivat sulfate salt in the disclosed mini tablet is in an amount that is equivalent to about 0.1 mg to about 5 mg of mitapivat). In other embodiments, the mitapivat sulfate salt in the disclosed mini tablet is in an amount that is equivalent to about 0.5 mg to about 2.0 mg of mitapivat. In another alternative, the mitapivat sulfate salt in the disclosed mini tablet is equivalent to about 1.0 mg of mitapivat. In other embodiments, the minitablet comprises a mitapivat sulfate hydrate salt (e.g., mitapivat hemisulfate sesquihydrate). In another alternative, the mitapivat sulfate hydrate salt (e.g., mitapivat hemisulfate sesquihydrate) in the disclosed mini tablet is present in an amount that is equivalent to about 0.1 mg to about 10.0 mg of mitapivat. In another alternative, the mitapivat sulfate hydrate salt (e.g., mitapivat hemisulfate sesquihydrate) in the disclosed mini-tablet is present in an amount that is equivalent to about 0.1 mg to about 3.0 mg of mitapivat. In yet another alternative, the mitapivat sulfate hydrate salt (e.g., mitapivat hemisulfate sesquihydrate) in the disclosed mini tablets is present in an amount that is equivalent to about 1.0 mg to about 1.5 mg of mitapivat. In yet another alternative, the mitapivat sulfate hydrate salt (e.g., mitapivat hemisulfate sesquihydrate) in the disclosed mini tablets is present in an amount that is equivalent to about 1.0 mg of mitapivat. In another aspect, the mitapivat sulfate hydrate salt in any of the foregoing alternatives in this paragraph is crystalline. In another aspect, the mitapivat sulfate hydrate salt in any of the foregoing alternatives in this paragraph is crystalline Form A.

In some embodiments, the minitablet comprises about 0.1 mg to about 5 mg of mitapivat or a pharmaceutically acceptable salt thereof in an amount that is equivalent to about 0.1 mg to about 5 mg of mitapivat. In some embodiments, the minitablet comprises about 1 mg of mitapivat or a pharmaceutically acceptable salt thereof in an amount that is equivalent to about 1 mg of mitapivat. In some embodiments, the minitablet comprises about 0.1 mg to about 5 mg of mitapivat or a pharmaceutically acceptable salt thereof in an amount that is equivalent to about 0.1 mg to about 5 mg of mitapivat and one or more inactive ingredients (e.g., a filler, disintegrant, lubricant, binder, diluent, and the like). In some embodiments, the minitablet comprises about 1 mg of mitapivat or a pharmaceutically acceptable salt thereof in an amount that is equivalent to about 1 mg of mitapivat and one or more inactive ingredients (e.g., a filler, disintegrant, lubricant, binder, diluent, and the like). In some embodiments, the minitablet comprises about 0.1 mg to about 5 mg of mitapivat or a pharmaceutically acceptable salt thereof in an amount that is equivalent to about 0.1 mg to about 5 mg of mitapivat, microcrystalline cellulose, mannitol, croscarmellose sodium and sodium stearyl fumarate and having as its longest dimension or diameter a length of about 10.0 mm to about 0.1 mm, wherein the minitablet is suitable for mixing with food before oral administration once or twice daily to patients with difficulties swallowing. In some embodiments, the minitablet comprises about 1 mg of mitapivat or a pharmaceutically acceptable salt thereof in an amount that is equivalent to about 1 mg of mitapivat, microcrystalline cellulose, mannitol, croscarmellose sodium and sodium stearyl fumarate and having as its longest dimension or diameter a length of about 10.0 mm to about 0.1 mm, wherein the minitablet is suitable for mixing with food before oral administration once or twice daily to patients with difficulties swallowing. In some embodiments, the minitablet comprises about 0.1 mg to about 5 mg of mitapivat or a pharmaceutically acceptable salt thereof in an amount that is equivalent to about 0.1 mg to about 5 mg of mitapivat, microcrystalline cellulose, mannitol, croscarmellose sodium and sodium stearyl fumarate and having as its longest dimension or diameter a length of about 10.0 mm to about 0.1 mm for oral administration once or twice daily to patients that are between about 1 year to about 12 years of age and weighing between about 7 kg to less than about 40 kg or adult patients with difficulties swallowing. In some embodiments, the minitablet comprises about 1 mg of mitapivat or a pharmaceutically acceptable salt thereof in an amount that is equivalent to about 1 mg of mitapivat, microcrystalline cellulose, mannitol, croscarmellose sodium and sodium stearyl fumarate and having as its longest dimension or diameter a length of about 10.0 mm to about 0.1 mm for oral administration once or twice daily to patients that are between about 1 year to about 12 years of age and weighing between about 7 kg to less than about 40 kg or to adult patients with difficulties swallowing.

In certain embodiments, the mitapivat or pharmaceutically acceptable salt thereof present in the disclosed mini tablet is about 1% to about 80% by weight. Alternatively, the mitapivat or pharmaceutically acceptable salt thereof present in the disclosed mini tablet is about 5% to about 50% by weight. In another alternative, the mitapivat or pharmaceutically acceptable salt thereof present in the disclosed mini tablet is about 10% to about 15% by weight. In certain embodiments, the pharmaceutically acceptable salt thereof present in the disclosed mini tablet in an amount of about 1% to about 80% by weight is a mitapivat sulfate salt, e.g., mitapivat sulfate hydrate, mitapivat hemisulfate sesquihydrate, mitapivat sulfate hydrate Form A, or mitapivat hemisulfate sesquihydrate Form A. In certain embodiments, the pharmaceutically acceptable salt thereof present in the disclosed mini tablet in an amount of about 5% to about 50% by weight is a mitapivat sulfate salt, e.g., mitapivat sulfate hydrate, mitapivat hemisulfate sesquihydrate, mitapivat sulfate hydrate Form A, or mitapivat hemisulfate sesquihydrate Form A. In certain embodiments, the pharmaceutically acceptable salt thereof present in the disclosed mini tablet in an amount of about 10% to about 15% by weight is a mitapivat sulfate salt, e.g., mitapivat sulfate hydrate, mitapivat hemisulfate sesquihydrate, mitapivat sulfate hydrate Form A, or mitapivat hemisulfate sesquihydrate Form A.

In one aspect, the microcrystalline cellulose in the disclosed mini tablet is present in an amount of about 5% to about 90% by weight. Alternatively, the microcrystalline cellulose in the disclosed mini tablet is present in an amount of about 55% to about 65% by weight.

In one aspect, croscarmellose in the disclosed mini tablet is present in an amount of about 1% to about 10% by weight. Alternatively, the croscarmellose sodium in the disclosed mini tablet is present in an amount of about 2% to about 4% by weight.

In one aspect, the sodium stearyl fumarate in the disclosed mini tablet is present in an amount of about 0.1% to about 5% by weight. In some embodiments, the sodium stearyl fumarate in the disclosed mini-tablets is present in an amount of about 1.5% to about 2.5% by weight.

In one aspect, the mannitol in the disclosed mini tablet is present in an amount of about 5% to about 90% by weight. Alternatively, the mannitol is present in an amount of about 20% to about 25% by weight.

As used herein, “Compound 1” and “mitapivat” are used interchangeably, referring to N-(4-(4-(cyclopropylmethyl)piperazine-1-carbonyl)phenyl)quinoline-8-sulfonamide.

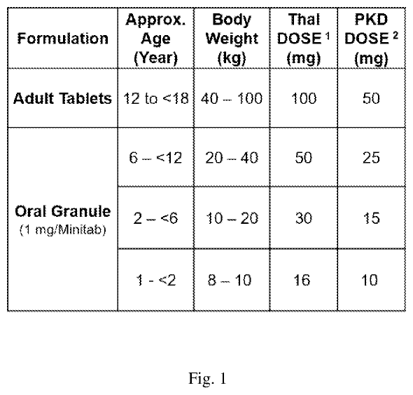

A “pharmaceutically effective amount” of the disclosed mini-tablets is an amount of one or more minitablets that would provide or deliver mitapivat or a pharmaceutically acceptable salt thereof in sufficient quantities to provide a therapeutic benefit in the treatment of a condition or to delay or minimize one or more symptoms associated with the condition. The term “pharmaceutically effective amount”, “therapeutically effective amount” and “effective amount” are used interchangeably. In one aspect, a pharmaceutically effective amount of a disclosed mini-tablet means an amount of a therapeutic agent, alone or in combination with other therapies, which provides a therapeutic benefit in the treatment of the condition. The term “pharmaceutically effective amount” can encompass an amount that improves overall therapy, reduces or avoids symptoms, signs, or causes of the condition, and/or enhances the therapeutic efficacy of another therapeutic agent. In certain embodiments, a pharmaceutically effective amount is an amount sufficient for eliciting measurable activation of wild-type or mutant PKR. In certain embodiments, a pharmaceutically effective amount is an amount sufficient for regulating 2,3-diphosphoglycerate levels in the blood of a patient in need thereof or for treating pyruvate kinase deficiency (PKD), hemolytic anemia (e.g., chronic hemolytic anemia, hereditary non-spherocytic anemia), sickle cell disease, thalassemia (e.g., alfa thalassemia, beta-thalassemia or non-transfusion-dependent thalassemia), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), anemia of chronic diseases or treating diseases or conditions that are associated with increased 2,3-diphosphoglycerate levels (e.g., liver diseases). In certain embodiments, a pharmaceutically effective amount is an amount sufficient for eliciting measurable activation of wild-type or mutant PKR and for regulating 2,3-diphosphoglycerate levels in the blood of a patient in need thereof or for treating pyruvate kinase deficiency (PKD), hemolytic anemia (e.g., chronic hemolytic anemia, hereditary non-spherocytic anemia), sickle cell disease, thalassemia (e.g., alfa thalassemia, beta-thalassemia or non-transfusion-dependent thalassemia), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), anemia of chronic diseases or treating diseases or conditions that are associated with increased 2,3-diphosphoglycerate levels (e.g., liver diseases). In one aspect, the pharmaceutically effective amount is the amount required to generate a subject's hemoglobin response of ≥1.0 g/dL (such as ≥1.5 g/dL or ≥2.0 g/dL) increase in Hb concentration from baseline. In one aspect, the subject's baseline Hb concentration is the average of all available Hb concentrations before treatment (e.g. all Hb concentrations obtained during about eight weeks to about one day prior to the treatment, all Hb concentrations obtained during about four weeks to about one day prior to the treatment, all Hb concentrations obtained during about one week to about one day prior to the treatment) with a compound or minitablet described herein. In certain aspects, the pharmaceutically effective amount is the amount required to reduce the patient's transfusion burden. The amount of the disclosed mini-tablets to be administered will vary depending upon the patient to be treated. For example, a specific dosage and treatment regimen for any particular patient will depend upon a variety of factors, including age, body weight, general health, sex, diet, time of administration, rate of excretion, drug combination, the judgment of the treating physician, and the severity of the particular disease being treated. In one aspect, the pharmaceutically effective amount is an amount of the mini-tablets that delivers between 0.01-100 mg/kg body weight/day of mitapivat, such as e.g., 0.1-100 mg/kg body weight/day. For a child 12 to 18 years old, in one aspect, a pharmaceutically effective amount of the mini-tablets is an amount that delivers about 50 to about 100 mg of mitapivat or, when administering mitapivat sulfate, the equivalent of about 50 to about 100 mg mitapivat; for a child 6 to 12 years old, in one aspect, pharmaceutically effective amount is an amount of the mini-tablets that delivers about 25 to about 50 mg of mitapivat or, when administering mitapivat sulfate, the equivalent of about 25 to about 50 mg mitapivat; for a child 2 to years old, in one aspect, pharmaceutically effective amount is an amount of the mini-tablets that delivers about 15 to about 30 mg of mitapivat or, when administering mitapivat sulfate, the equivalent of 15-30 mg mitapivat; or for a child 1 to years old, in one aspect, pharmaceutically effective amount is an amount of the mini-tablets that delivers about 10 to about 16 mg of mitapivat or, when administering mitapivat sulfate, the equivalent of about 10 to about 16 mg mitapivat. For a subject with a body weight between about 40 kg to about 100 kg, in one aspect, pharmaceutically effective amount is an amount of the mini-tablets that delivers about 50 mg to about 100 mg of mitapivat or, when administering mitapivat sulfate, the equivalent of about 50 mg to about 100 mg mitapivat; for a subject with a body weight between about 20 to about 40 kg, in one aspect, pharmaceutically effective amount is an amount of the mini-tablets that delivers about 25 mg to about 50 mg of mitapivat or, when administering mitapivat sulfate, the equivalent of about 25 mg to about 50 mg mitapivat; for a subject with a body weight between about 10 kg to about 20 kg, in one aspect, pharmaceutically effective amount is an amount of the mini-tablets that delivers about 15 mg to about 30 mg of mitapivat or, when administering mitapivat sulfate, the equivalent of about 15 mg to about 30 mg mitapivat; or for a subject with a body weight between about 8 kg to about 10 kg, in one aspect, pharmaceutically effective amount is an amount of the mini-tablets that delivers about 10 mg to about 16 mg of mitapivat or, when administering mitapivat sulfate, the equivalent of 10 mg to about 16 mg mitapivat. In some embodiments, suitable doses are as described in FIG. 1 or in Table 3. In one embodiment, a suitable dose of a minitablet described herein comprises administering one or more minitablets each having about 1 mg of mitapivat or a pharmaceutically acceptable salt thereof (e.g., mitapivat hemisulfate sesquihydrate) in an amount equivalent to 1 mg of mitapivat so as to provide the recommended dosage amount to a subject at the corresponding age and weight listed in Table 3.

As used herein, reduction in transfusion burden means at least 10% reduction in the number of RBC units transfused within at least 2 weeks of treatment (compared with historical transfusion burden standardized to the same period). In some embodiments, the reduction in transfusion burden is at least 20% reduction in the number of RBC units transfused within at least 3 weeks of treatment. In certain embodiments, the reduction in transfusion burden is ≥33% reduction in the number of RBC units transfused within at least 5 weeks of treatment. In certain embodiments, reduction of transfusion burden is ≥33% reduction in the number of RBC units transfused within at least 10 weeks (e.g., at least 20 weeks or at least 24 weeks) of treatment. As used herein, reduction in transfusion burden means at least 50% reduction in the number of RBC units transfused within at least 5 weeks of treatment (compared with historical transfusion burden standardized to the same period). As used herein, reduction in transfusion burden means at least 50% reduction in the number of RBC units transfused within at least 10 weeks (e.g., at least 20 weeks or at least 24 weeks) of treatment.

As used herein, sickle cell disease (SCD), Hemoglobin SS disease, and sickle cell anemia are used interchangeably. Sickle cell disease (SCD) is an inherited blood disorder caused by the presence of sickle hemoglobin (HbS). In certain embodiments, subjects with SCD have abnormal hemoglobin, called hemoglobin S or sickle hemoglobin, in their red blood cells. In certain embodiments, people having SCD have at least one abnormal genes causing the body to make hemoglobin S. In certain embodiments, people having SCD have two hemoglobin S genes, Hemoglobin SS. In certain embodiments, people with SCD have chronic hemolytic anemia interrupted by acute and recurrent clinical events, for example, the most common being acute pain generally referred to as “acute vaso-occlusive crises (VOC).

Thalassemia is an inherited blood disorder in which the normal ratio of α- to β-globin production is disrupted due to a disease-causing variant in 1 or more of the globin genes. In certain embodiments, Alpha-globin aggregates (as found in β-thalassemia) readily precipitate, which disrupts the red blood cell (RBC) membrane and results in oxidative stress. In certain embodiments, Beta-globin tetramers (Hb H, found in α-thalassemia) are generally more soluble, but are still unstable and can form precipitates. The imbalance of the globin chain synthesis can lead to a net reduction in Hb concentrations and has dramatic effects on the survival of RBC precursors, ultimately resulting in their premature destruction in the bone marrow and in extramedullary sites (Cappellini et al, 2014). In certain embodiments, the disorder results in large numbers of red blood cells being destroyed, which leads to anemia. In certain embodiments, the thalassemia is alpha thalassemia. In certain embodiments, the thalassemia is beta thalassemia. In other embodiments, the thalassemia is non-transfusion-dependent thalassemia. In other embodiments, the thalassemia is beta thalassemia intermedia. In other embodiments, the thalassemia is Hb E beta thalassemia. In other embodiments, the thalassemia is beta thalassemia with mutations of 1 or more alfa genes.

The term “activating” as used herein means an agent that (measurably) increases the activity of wild type pyruvate kinase R (wt PKR) or causes wild type pyruvate kinase R (wt PKR) activity to increase to a level that is greater than wt PKR's basal levels of activity or an agent that (measurably) increases the activity of a mutant pyruvate kinase R (mPKR) or causes mutant pyruvate kinase R (mPKR) activity to increase to a level that is greater than that mutant PKR's basal levels of activity, for examples, to a level that is 20%, 40%, 50%, 60%, 70%, 80%, 90% or 100% of the activity of wild type PKR.

The term “packed red blood cells” or PRBCs as used herein refer to red blood cells made from a unit of whole blood by centrifugation and removal of most of the plasma. In certain embodiments, a PRBC unit has a hematocrit of at least about 95%. In certain embodiments, a PRBC unit has a hematocrit of at least about 90%. In certain embodiments, a PRBC unit has a hematocrit of at least about 80%. In certain embodiments, a PRBC unit has a hematocrit of at least about 70%. In certain embodiments, a PRBC unit has a hematocrit of at least about 60%. In certain embodiments, a PRBC unit has a hematocrit of at least about 50%. In certain embodiments, a PRBC unit has a hematocrit of at least about 40%. In certain embodiments, a PRBC unit has a hematocrit of at least about 30%. In certain embodiments, a PRBC unit has a hematocrit of at least about 20%. In certain embodiments, a PRBC unit has a hematocrit of at least about 10%.

The terms “treatment,” “treat,” and “treating” refer to reversing, alleviating, reducing the likelihood of developing, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to reduce the likelihood of or delay their recurrence.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment. In certain embodiments, the term “subject” refers to a human subject in need of treatment of a disease. In certain embodiments, the term “subject” refers to a human subject in need of treatment of PKD. In certain embodiments, the term “subject” refers to a human subject in need of treatment of thalassemia. In certain embodiments, the term “subject” refers to a human subject in need of treatment of sickle cell disease. In certain embodiments, the term “subject” refers to a human subject in need of treatment of hemolytic anemia. In certain embodiments, the term “subject” refers to a human adult over 18 years old in need of treatment of a disease. In certain embodiments, the term “subject” refers to a human child no more than 18 years old in need of treatment of a disease. In another aspect, “subject” refers to patients requiring small doses and/or who have difficulty swallowing, e.g., to a pediatric patient, an elderly patient or a patient with an illness that effects swallowing. “Pediatric patient” or “pediatric subject” refers to a child of about one-year old to about two-years old; two-year old to about six-year old; six-year old to about twelve-year old; or twelve-year old to about eighteen-year old. In certain embodiments, the pediatric patient is between about 1 year of age to about 12 years of age. In other certain embodiments, the pediatric patient is between about 2 years of age to about 12 years of age. In still other certain embodiments the pediatric patient is between about 1 year of age to less than about 2 years of age. “Elderly patient” refers to a patient over 65 years old; over 70 years old; over 75 years old; or over 80 years old.

In certain embodiments, the subject is a patient in need of regular blood transfusion. As used here, the regular blood transfusion refers to at least 4 transfusion episodes in a 52-week period prior to the treatment. In certain embodiments, the regular blood transfusion refers to at least 5 transfusion episodes in a 52-week period prior to the treatment. In certain embodiments, the regular blood transfusion refers to at least 6 transfusion episodes in a 52-week period prior to the treatment. In certain embodiments, the regular blood transfusion refers to at least 7 transfusion episodes in a 52-week period prior to the treatment. In certain embodiments, the subject with a least one of the indications selected from the sickle cell disease, thalassemia, PKD under regular transfusion, and non-transfusion dependent PKD, has not been exposed to sotatercept (ACE-011), luspatercept (ACE-536), ruxolitinib, or gene therapy. In certain embodiments, such subject is not taking inhibitors of cytochrome P450 (CYP)3A4, strong inducers of CYP3A4, strong inhibitors of P-glycoprotein (P-gp), or digoxin. In certain embodiments, such subject is not receiving chronic anticoagulant therapy, anabolic steroids, hematopoietic stimulating agents (eg, erythropoietins, granulocyte colony stimulating factors, thrombopoietins), or allergic to sulfonamides.

Unless otherwise specified to the contrary, when a range is disclosed in connection to any of the elements described herein both the endpoints and the values in-between the endpoints are to be included in that range. For example, “about 1 year of age to about 12 years of age” includes about 1 year of age and about 12 years of age, and any and all values in between. When an endpoint of a range is preceded by the term “less than” or “less than about” it will be understood that such an endpoint is not included. For example, “a body weight of about 20 kg to less than about 40 kg” includes a body weight of about 20 kg and any and all values up to, but not including 40 kg.

As used herein, the terms “about” and “approximately” when used in combination with a numeric value or range of values means the value or range of values may deviate to an extent deemed reasonable to one of ordinary skill in the art.

### Methods of Treatment and Uses of Compounds and Compositions

In one embodiment, the disclosed mini-tablets are administered by combining and/or mixing with a food vehicle to mask the bitter taste of mitapivat or one or more of its pharmaceutically acceptable salts. The mini-tablets can be combined and/or mixed whole with the food vehicle, or, alternatively, crushed and mixed, preferably into a homogenous mixture. To provide a homogenous mixture, the food vehicle is mixed with mitapivat or one or more of its pharmaceutically acceptable salts and administered to the patient instantly or within about two hours (e.g. within about fifteen minutes or within about 30 minutes) after mixing.

Examples of suitable food vehicles include strawberry jam, peanut butter, chocolate pudding, strawberry yogurt, apple juice, apple sauce, milk, yogurt, and soft grain-based cereal (such as cream of rice cereal). In one embodiment, the food vehicle is chocolate pudding, strawberry jam, peanut butter, strawberry yogurt, or apple sauce. In another aspect, the food vehicle is chocolate pudding, strawberry jam, or peanut butter. In another embodiment, the food vehicle is strawberry yogurt and apple sauce.

In another embodiment, the disclosed minitablets are taken with water.

In one aspect, the mitapivat used in the disclosed mini-tablet allosteric activators of PKR, and are generally useful for treating the underlying condition of PKD.

Thus, provided herein are methods of treating Pyruvate Kinase Deficiency (PKD) in a subject (e.g., a pediatric patient, an elderly patient or a patient with a disease or condition that makes swallowing difficult) in need thereof, comprising administering to the subject a pharmaceutically effective amount of the disclosed mini-tablet. Also provided is the disclosed mini-tablet for use in treating Pyruvate Kinase Deficiency (PKD) in a subject (e.g., a pediatric patient, an elderly patient or a patient with a disease or condition that makes swallowing difficult) in need thereof. Further provided is the use of the disclosed mini-tablet in the manufacture of a medicament for treating Pyruvate Kinase Deficiency (PKD). Exemplified conditions related to PKD include, but are not limited to, anemias, cholecystolithiasis, gallstones, tachycardia, hemochromatosis, icteric sclera, splenomegaly, leg ulcers, jaundice, fatigue, and shortness of breath. As described herein, PKD is a deficiency of PKR. In certain embodiments, the deficiency of PKR is associated with a PKR mutation.

Pyruvate kinase deficiency (PKD) is a glycolytic enzymopathy that results in life-long hemolytic anemia. In certain embodiments, the subject (e.g., a pediatric patient, an elderly patient or a patient with a disease or condition that makes swallowing difficult) having PKD is a patient having at least 2 mutant alleles in PKLR gene. In certain embodiments, the subject having PKD is a patient having at least 2 mutant alleles in PKLR gene and at least one is a missense mutation. See Canu. et. al, Blood Cells, Molecules and Diseases 2016, 57, pp. 100-109. In certain embodiments, a subject having PKD has an Hb concentration less than or equal to 10.0 g/dL. In certain embodiments, the subject having PKD is an adult not under regular transfusion (e.g. having had no more than 4 transfusion episodes in the 12-month period up to the treatment). In certain embodiments, the subject having PKD is an adult transfusion independent (e.g. having no more than 4 units of RBCs transfused in the 12-month period prior to the treatment). In certain embodiments, the subject having PKD is an adult transfusion independent having no more than 4 units of RBCs transfused in the 12-month period prior to the treatment and no transfusion in the 3-month period prior to the treatment. In certain embodiments, the subject having PKD is an adult under regular transfusion (e.g. having had at least 4 transfusion episodes (e.g., at least 5 transfusion episodes or at least 6 transfusion episodes) in the 12-month period prior to the treatment). In certain embodiments, the subject having PKD has a total number of at least 5 transfusion episodes during the subject's lifetime. In certain embodiments, the subject having PKD has a total number of at least 10 transfusion episodes during the subject's lifetime. In certain embodiments, the subject having PKD has a total number of at least 15 transfusion episodes during the subject's lifetime. In certain embodiments, the subject having PKD has a total number of at least 20 transfusion episodes during the subject's lifetime. In certain embodiments, the subject having PKD has a total number of at least 25 transfusion episodes during the subject's lifetime. In certain embodiments, the subject having PKD has a total number of at least 30 transfusion episodes during the subject's lifetime. In certain embodiments, the subject having PKD has a total number of at least 40 transfusion episodes during the subject's lifetime. In certain embodiments, the subject having PKD has a total number of at least 50 transfusion episodes during the subject's lifetime. In certain embodiments, the subject having PKD has a total number of at least 60 transfusion episodes during the subject's lifetime. In certain embodiments, the subject having PKD has a total number of at least 70 transfusion episodes during the subject's lifetime. In certain embodiments, the subject having PKD is not homozygous for the R479H mutation or does not have 2 non-missense mutations in the PKLR gene. In certain embodiments, the subject having PKD, under regular transfusion, has hemoglobin (Hb) ≤12.0 g/dL (if male) or ≤11.0 g/dL (if female), prior to the treatment. In certain embodiments, the subject having PKD, under regular transfusion, has transfusion occurring on average less than or equal to once every three weeks. In certain embodiments, the subject having PKD has received at least 0.8 mg (e.g. at least 1.0 mg) folic acid daily (e.g. for at least 21 days) prior to the treatment. In certain embodiments, the subject with PKD under regular transfusion achieves a reduction in transfusion burden (e.g. at least 33% reduction in the number of RBC units transfused) during the at least 5 weeks, at least 10 weeks, at least 15 weeks, at least 20 weeks, at least 24 weeks, at least 28 weeks, or at least 32 weeks of treatment compared with the historical transfusion burden. In certain embodiments, the subject having PKD, not under regular transfusion (having had no more than 4 transfusion episodes in the 12-month period prior to the treatment and/or no transfusion in the 3 months prior to the treatment), has hemoglobin (Hb) ≤10.0 g/dL regardless of gender prior to the treatment. In certain embodiments, the subject having PKD has undergone splenectomy. In certain embodiments, the PKD subject is not homozygous for R479H mutation or does not have 2 non-missense mutation in the PKLR gene.

In certain embodiments, the subject with PKD achieves a hemoglobin response of at least 0.5 g/dL increase in Hb concentration after the treatment compared to the baseline of prior to the treatment. In certain embodiments, the subject with PKD achieves a hemoglobin response of at least 1.0 g/dL increase in Hb concentration after the treatment compared to the baseline of prior to the treatment. In certain embodiments, the subject with PKD achieves a hemoglobin response of at least 1.5 g/dL increase in Hb concentration from baseline prior to the treatment. In certain embodiments, the subject with PKD achieves a hemoglobin response of at least 2.0 g/dL increase in Hb concentration from baseline prior to the treatment.

In an embodiment, the mutant PKR is selected from G332S, G364D, T384M, K410E, R479H, R479K, R486W, R532W, R510Q, and R490W. In certain embodiments, the mutant PKR is selected from A468V, A495V, I90N, T408I, and Q421K, and R498H. In certain embodiments, the mutant PKR is R532W, K410E, or R510Q. In certain embodiments, the mutant PKR is R510Q, R486W.

In other aspects, provided are methods of treating a disease selected from hemolytic anemia, sickle cell disease, thalassemia, hereditary spherocytosis, hereditary elliptocytosis, abetalipoproteinemia, Bassen-Kornzweig syndrome, and paroxysmal nocturnal hemoglobinuria in a subject (e.g., a pediatric patient, an elderly patient or a patient with a disease or condition that makes swallowing difficult) in need thereof, comprising administering to the subject a pharmaceutically effective amount of the disclosed mini-tablet. Also provided is the disclosed mini-tablet for use in treating disease selected from hemolytic anemia, sickle cell disease, thalassemia, hereditary spherocytosis, hereditary elliptocytosis, abetalipoproteinemia, Bassen-Kornzweig syndrome, and paroxysmal nocturnal hemoglobinuria in a subject (e.g., a pediatric patient, an elderly patient or a patient with a disease or condition that makes swallowing difficult). Further provided is the use of the disclosed mini-tablet in the manufacture of a medicament for treating a disease selected from hemolytic anemia, sickle cell disease, thalassemia, hereditary spherocytosis, hereditary elliptocytosis, abetalipoproteinemia, Bassen-Kornzweig syndrome, and paroxysmal nocturnal hemoglobinuria in a subject (e.g., a pediatric patient, an elderly patient or a patient with a disease or condition that makes swallowing difficult) in need thereof. In one aspect, the disease to be treated is hemolytic anemia.

In other aspects, provided herein are methods for treating thalassemia in a subject (e.g., a pediatric patient, an elderly patient or a patient with a disease or condition that makes swallowing difficult) in need thereof, comprising administering to the subject a pharmaceutically effective amount of the disclosed mini-tablets. Also provided is the disclosed mini-tablets for use in treating thalassemia. Further provided is the disclosed mini-tablets in the manufacture of a medicament for treating thalassemia.

In certain embodiments, the subject is a subject with thalassemia. In certain embodiments, the subject has thalassemia such as β-thalassemia intermedia, Hb E β-thalassemia, α-thalassemia (Hb H disease), or β-thalassemia with mutations of 1 or more α genes. In some embodiments, the thalassemia subject does not have Hb S or Hb C forms of thalassemia. In some embodiments, the thalassemia subject has not had splenectomy. In certain embodiments, the subject has beta-thalassemia or non-transfusion-dependent thalassemia. In certain embodiments, the subject is an adult subject. In certain embodiments, the subject has beta-thalassemia. In certain embodiments, the subject has alpha-thalassemia. In certain embodiments, the subject has a hemoglobin concentration of less than or equal to 6.0 g/dL. In certain embodiments, the subject has a hemoglobin concentration of less than or equal to 7.0 g/dL. In certain embodiments, the subject has a hemoglobin concentration of less than or equal to 8.0 g/dL. In certain embodiments, the subject has a hemoglobin concentration of less than or equal to 9.0 g/dL. In certain aspects, the subject having non-transfusion-dependent thalassemia does not have a known history (e.g., has been diagnosed in the past) of Hb S or Hb C forms of thalassemia. In certain embodiments, the term “non-transfusion dependent” thalassemia refers to subjects with thalassemia having no more than 4 (e.g. five) units of RBCs transfused during a 24-week period up to the first day of administration of a mini-tablet described herein and/or no RBC transfusions in the 8 weeks prior to the first day of administration of a mini-tablet described herein. In certain aspects, the subject is a transfusion dependent thalassemia subject. In certain embodiments, the term “transfusion dependent” thalassemia refers to subjects with thalassemia having 5 to 30 RBC units transfused during the 24-week period before the treatment. In some embodiments, the transfusion dependent thalassemia subject has 6 to 20 RBC units transfused during the 24-week period before the treatment. In some embodiments, the transfusion dependent thalassemia subject has 6 to 20 RBC units transfused during the 24-week period up to the first day of administration of a mini-tablet described herein and/or no RBC transfusions in the 8 weeks prior to the first day of administration of a mini-tablet described herein. In certain embodiments, the subject with non-transfusion dependent thalassemia achieves a hemoglobin response of at least 0.5 g/dL increase in Hb concentration after the treatment compared to the baseline of prior to the treatment. In certain embodiments, the subject with non-transfusion dependent thalassemia achieves a hemoglobin response of at least 1.0 g/dL increase in Hb concentration after the treatment compared to the baseline of prior to the treatment. In certain embodiments, the subject with non-transfusion dependent thalassemia achieves a hemoglobin response of at least 1.5 g/dL increase in Hb concentration from baseline prior to the treatment. In certain embodiments, the subject with non-transfusion dependent thalassemia achieves a hemoglobin response of at least 2.0 g/dL increase in Hb concentration from baseline prior to the treatment. In certain embodiments, the subject with transfusion dependent thalassemia achieves a hemoglobin response of at least 0.5 g/dL increase in Hb concentration after the treatment compared to the baseline of prior to the treatment. In certain embodiments, the subject with transfusion dependent thalassemia achieves a hemoglobin response of at least 1.0 g/dL increase in Hb concentration after the treatment compared to the baseline of prior to the treatment. In certain embodiments, the subject with transfusion dependent thalassemia achieves a hemoglobin response of at least 1.5 g/dL increase in Hb concentration from baseline prior to the treatment. In certain embodiments, the subject with transfusion dependent thalassemia achieves a hemoglobin response of at least 2.0 g/dL increase in Hb concentration from baseline prior to the treatment.

In other aspects, provided herein are methods for increasing the lifetime of red blood cells (RBCs) in a subject (e.g., a pediatric patient, an elderly patient or a patient with a disease or condition that makes swallowing difficult) in need thereof comprising administering to the subject a pharmaceutically effective amount of the disclosed mini-tablets. In such aspects the subject has a disease or condition such as PKD, sickle cell disease, thalassemia or anemia. Also provided is the disclosed mini-tablets for use in increasing the lifetime of red blood cells (RBCs) in a subject (e.g., a pediatric patient, an elderly patient or a patient with a disease or condition that makes swallowing difficult) in need thereof. In such aspects the subject has a disease or condition such as PKD, sickle cell disease, thalassemia or anemia. Further provided is the use of the disclosed mini-tablets in the manufacture of a medicament for increasing the lifetime of red blood cells (RBCs). In one aspect, the disclosed mini-tablets are added directly to whole blood or packed red blood cells extracorporeally.

In other aspects, provided herein are methods for regulating 2,3-diphosphoglycerate levels in the blood of a subject (e.g., a pediatric patient, an elderly patient or a patient with a disease or condition that makes swallowing difficult) in need thereof comprising contacting blood with a pharmaceutically effective amount of the disclosed mini-tablets. In such aspects the subject has a disease or condition such as PKD, sickle cell disease, thalassemia or anemia. Also provided is the disclosed mini-tablets for use in regulating 2,3-diphosphoglycerate levels in the blood of a subject (e.g., a pediatric patient, an elderly patient or a patient with a disease or condition that makes swallowing difficult) in need thereof. In such aspects the subject has a disease or condition such as PKD, sickle cell disease, thalassemia or anemia. Further provided is the use of the disclosed mini-tablets in the manufacture of a medicament for regulating 2,3-diphosphoglycerate levels in the blood of a subject. (e.g., a pediatric patient, an elderly patient or a patient with a disease or condition that makes swallowing difficult).

In other aspects, provided herein are methods for treating anemia in a subject (e.g., a pediatric patient, an elderly patient or a patient with a disease or condition that makes swallowing difficult) in need thereof comprising administering to the subject a pharmaceutically effective amount of the disclosed mini-tablets. Also provided is the disclosed mini-tablets for use in treating anemia in a subject (e.g., a pediatric patient, an elderly patient or a patient with a disease or condition that makes swallowing difficult) in need thereof. Further provided is the use of the disclosed mini-tablets in the manufacture of a medicament for treating anemia. In one aspect, the anemia to be treated is dyserythropoietic anemia.

In certain embodiments, the anemia is a dyserythropoietic anemia such as congenital dyserythropoietic anemia type I, II, III, or IV. In certain embodiments, the anemia is hemolytic anemia. In certain embodiments, the hemolytic anemia is a congenital and/or hereditary form of hemolytic anemia such as PKD, sickle cell disease, thalassemias (e.g. alpha or beta thalassemia), hereditary spherocytosis, hereditary elliptocytosis), paroxysmal nocturnal hemoglobinuria, abeta-liproteinemia (Bassen-Kornzweig syndrome). In certain embodiments, the hemolytic anemia is acquired hemolytic anemia such as autoimmune hemolytic anemia, drug-induced hemolytic anemia. In certain embodiments, the hemolytic anemia is anemia as part of a multi-system disease, such as the anemia of Congenital Erythropoietic Purpura, Fanconi, Diamond-Blackfan.

As used herein, the term “anemia” refers to a deficiency of red blood cells (RBCs) and/or hemoglobin. As used herein, anemia includes all types of clinical anemia, for example (but not limited to): microcytic anemia, iron deficiency anemia, hemoglobinopathies, heme synthesis defect, globin synthesis defect, sideroblastic defect, normocytic anemia, anemia of chronic disease, aplastic anemia, hemolytic anemia, macrocytic anemia, megaloblastic anemia, pernicious anemia, dimorphic anemia, anemia of prematurity, Fanconi anemia, hereditary spherocytosis, sickle cell disease, warm autoimmune hemolytic anemia, cold agglutinin hemolytic anemia, osteopetrosis, thalassemia, and myelodysplastic syndrome.

In certain embodiments, anemia can be diagnosed on a complete blood count. In certain embodiments, anemia can be diagnosed based on the measurement of one or more markers of hemolysis (e.g. RBC count, hemoglobin, reticulocytes, schistocytes, lactate Dehydrogenase (LDH), haptoglobin, bilirubin, and ferritin) and/or hemosiderinuria mean corpuscular volume (MCV) and/or red cell distribution width (RDW). In the context of the present invention, anemia is present if an individual has a hemoglobin (Hb) less than the desired level, for example, the Hb concentration of less than 14 g/dL, more preferably of less than 13 g/dL, more preferably of less than 12 g/dL, more preferably of less than 11 g/dL, or most preferably of less than 10 g/dL.

In certain embodiments, provided herein is a method of increasing the amount of hemoglobin in a subject (e.g., a pediatric patient, an elderly patient or a patient with a disease or condition that makes swallowing difficult) by administering a pharmaceutically effective amount of the disclosed mini-tablets as described herein. In such aspects the subject has a disease or condition such as PKD, sickle cell disease, thalassemia or anemia. In certain embodiments, also provided herein is a method of increasing the amount of hemoglobin in a subject (e.g., a pediatric patient, an elderly patient or a patient with a disease or condition that makes swallowing difficult) having thalassemia comprising administering to the subject a pharmaceutically effective amount of the disclosed mini-tablets. Further provided is a method of increasing the amount of hemoglobin in subjects (e.g., a pediatric patient, an elderly patient or a patient with a disease or condition that makes swallowing difficult) having thalassemia comprising administering a pharmaceutically effective amount of the disclosed mini-tablets as described herein to the subject. In certain embodiments, the provided methods increase hemoglobin concentration in the subject. In certain embodiments, the provided methods increase Hb concentration to a desired level, for example, above 10 g/dL, more preferably above 11 g/dL, more preferably above 12 g/dL, more preferably above 13 g/dL, or most preferably above 14 g/dL. In certain embodiments, the provided methods increase Hb concentration by at least about 0.5 g/dL. In certain embodiments, the provided methods increase Hb concentration by at least about 1.0 g/dL. In certain embodiments, the provided methods increase Hb concentration by at least about 1.5 g/dL. In certain embodiments, the provided methods increase Hb concentration by at least about 2.0 g/dL. In certain embodiments, the provided methods increase Hb concentration by at least about 2.5 g/dL. In certain embodiments, the provided methods increase Hb concentration by at least about 3.0 g/dL. In certain embodiments, the provided methods increase Hb concentration by at least about 3.5 g/dL. In certain embodiments, the provided methods increase Hb concentration by at least about 4.0 g/dL. In certain embodiments, the provided methods increase Hb concentration by at least about 4.5 g/dL. In certain embodiments, the provided methods increase Hb concentration by at least about 5.0 g/dL. In certain embodiments, the provided methods increase Hb concentration by at least about 5.5 g/dL. In certain embodiments, the provided methods increase Hb concentration by at least about 6.0 g/dL. In certain embodiments, the increase in Hb concentration is determined from baseline at one or more assessment between week 1 and week 20 (e.g., between week 2 and week 15, between week 3 and week 15, and between week 4 and week 12) of treatment with a pharmaceutically effective amount of the disclosed mini-tablets as described herein. In certain embodiments, the provided methods increase Hb concentration as described above in female subjects having thalassemia. In certain embodiments, the provided methods increase Hb concentration from baseline to about 12 g/dL in female subjects having thalassemia. In certain embodiments, the provided methods increase Hb concentration as described above in male subjects having thalassemia. In certain embodiments, the provided methods increase Hb concentration from baseline to about 13 g/dL in male subjects having thalassemia.

In some aspects, provided herein are methods for treating hemolytic anemia in a subject (e.g., a pediatric patient, an elderly patient or a patient with a disease or condition that makes swallowing difficult) in need thereof comprising administering to the subject a pharmaceutically effective amount of the disclosed mini-tablets. Also provided is the disclosed mini-tablets for use in treating hemolytic anemia in a subject (e.g., a pediatric patient, an elderly patient or a patient with a disease or condition that makes swallowing difficult) in need thereof. Further provided is the use of the disclosed mini-tablets thereof in the manufacture of a medicament for treating hemolytic anemia. In one aspect, the hemolytic anemia to be treated is hereditary and/or congenital hemolytic anemia, acquired hemolytic anemia, or anemia as part of a multi-system disease.

In some aspects, provided herein are methods for treating sickle cell disease in a subject (e.g., a pediatric patient, an elderly patient or a patient with a disease or condition that makes swallowing difficult) in need thereof comprising administering to the subject a pharmaceutically effective amount of the disclosed mini-tablets. Also provided is the disclosed mini-tablets for use in treating sickle cell disease in a subject (e.g., a pediatric patient, an elderly patient or a patient with a disease or condition that makes swallowing difficult) in need thereof. Further provided is the use of the disclosed mini-tablets thereof in the manufacture of a medicament for treating sickle cell disease.

In some aspects, provided herein are methods for treating thalassemia, hereditary spherocytosis, hereditary elliptocytosis, abetalipoproteinemia or Bassen-Kornzweig syndrome, sickle cell disease, paroxysmal nocturnal hemoglobinuria, acquired hemolytic anemia, or anemia of chronic diseases in a subject (e.g., a pediatric patient, an elderly patient or a patient with a disease or condition that makes swallowing difficult) in need thereof comprising administering to the subject a pharmaceutically effective amount of the disclosed mini-tablets. Also provided is the disclosed mini-tablets for use in treating thalassemia, hereditary spherocytosis, hereditary elliptocytosis, abetalipoproteinemia or Bassen-Kornzweig syndrome, sickle cell disease, paroxysmal nocturnal hemoglobinuria, acquired hemolytic anemia, or anemia in a subject (e.g., a pediatric patient, an elderly patient or a patient with a disease or condition that makes swallowing difficult) in need thereof. Further provided is the use of the disclosed mini-tablets in the manufacture of a medicament for treating thalassemia, hereditary spherocytosis, hereditary elliptocytosis, abetalipoproteinemia or Bassen-Kornzweig syndrome, sickle cell disease, paroxysmal nocturnal hemoglobinuria, acquired hemolytic anemia, or anemia.

In some aspects, provided herein are methods for activating wild-type or mutant PKR in red blood cells in a subject (e.g., a pediatric patient, an elderly patient or a patient with a disease or condition that makes swallowing difficult) in need thereof comprising administering to the subject a pharmaceutically effective amount of the disclosed mini-tablets. Also provided is the disclosed mini-tablets for use in activating wild-type or mutant PKR in red blood cells in a subject (e.g., a pediatric patient, an elderly patient or a patient with a disease or condition that makes swallowing difficult) in need thereof. Further provided is the use of the disclosed mini-tablets in the manufacture of a medicament for activating wild-type or mutant PKR in red blood cells.

In certain embodiments, the disclosed mini-tablets deliver sufficient quantities of mitapivat or a pharmaceutically acceptable salt thereof, to activate PKR mutants having lower activities compared to the wild type, and thus the minitablets are useful for methods of the present disclosure. Such mutations in PKR can affect enzyme activity (catalytic efficiency), regulatory properties (modulation by fructose bisphosphate (FBP)/ATP), and/or thermostability of the enzyme. Examples of such mutations are described in Valentini et al, JBC 2002.

In certain embodiments, the disclosed mini-tablets deliver sufficient quantities of mitapivat or a pharmaceutically acceptable salt thereof, to increase the affinity of PKR to phosphoenolpyruvate (PEP). In certain embodiments, the disclosed mini-tablets provide or deliver sufficient quantities of mitapivat or a pharmaceutically acceptable salt thereof to restore the ability of RBCs to cover PEP and ADP to pyruvate and ATP.

In certain embodiments, provided herein are methods of reducing transfusion frequency of a subject (e.g., a pediatric patient, an elderly patient or a patient with a disease or condition that makes swallowing difficulty) with PKD comprising administering to the subject the disclosed mini-tablets. In certain embodiments, the disclosed mini-tablets are administered. In certain embodiments, the transfusion frequency is reduced by at least 5% in the number of RBC units transfused over at least 15 weeks (compared with the historical transfusion burden standardized to the same period). In certain embodiments, the transfusion frequency is reduced by at least 10% in the number of RBC units transfused over at least 15 weeks. In certain embodiments, the transfusion frequency is reduced by at least 15% in the number of RBC units transfused over at least 15 weeks. In certain embodiments, the transfusion frequency is reduced by at least 20% in the number of RBC units transfused over at least 15 weeks. In certain embodiments, the transfusion frequency is reduced by at least 25% in the number of RBC units transfused over at least 15 weeks. In certain embodiments, the transfusion frequency is reduced by at least 30% in the number of RBC units transfused over at least 15 weeks. In certain embodiments, the transfusion frequency is reduced by at least 35% in the number of RBC units transfused over at least 15 weeks. In certain embodiments, the transfusion frequency is reduced by at least 40% in the number of RBC units transfused over at least 20 weeks. In certain embodiments, the transfusion frequency is reduced by at least 5% in the number of RBC units transfused over at least 20 weeks. In certain embodiments, the transfusion frequency is reduced by at least 10% in the number of RBC units transfused over at least 20 weeks. In certain embodiments, the transfusion frequency is reduced by at least 15% in the number of RBC units transfused over at least 20 weeks. In certain embodiments, the transfusion frequency is reduced by at least 20% in the number of RBC units transfused over at least 20 weeks. In certain embodiments, the transfusion frequency is reduced by at least 25% in the number of RBC units transfused over at least 20 weeks. In certain embodiments, the transfusion frequency is reduced by at least 30% in the number of RBC units transfused over at least 20 weeks. In certain embodiments, the transfusion frequency is reduced by at least 35% in the number of RBC units transfused over at least 20 weeks. In certain embodiments, the transfusion frequency is reduced by at least 40% in the number of RBC units transfused over at least 20 weeks.

Is some aspects, provided herein are methods of evaluating a subject (e.g., a pediatric patient, an elderly patient or a patient with a disease or condition that makes swallowing difficult), the method comprising: administering to the subject the disclosed mini-tablets; and acquiring a value for the level of mitapivat, the level of 2,3-diphosphoglycerate (2,3-DPG), the level of adenosine triphosphate (ATP), or the activity of PKR in the subject, to thereby evaluate the subject. In some aspects, the value for the level is acquired by analyzing the plasma concentration of mitapivat. In some aspects, the level of 2,3-DPG is acquired by analyzing the blood concentration of 2,3-DPG. In some aspects, the level of ATP is acquired by analyzing the blood concentration of ATP. In some aspects, the activity of PKR is acquired by analyzing the blood concentration of a ¹³C-label in the blood. In some aspects, the analysis is performed by sample analysis of bodily fluid. In some aspects, the bodily fluid is blood. In some aspects, the analysis is performed by mass spectroscopy. In some aspects, the analysis is performed by LC-MS.

In some aspects, provided herein are methods of evaluating a subject (e.g., a pediatric patient, an elderly patient or a patient with a disease or condition that makes swallowing difficult), the method comprising acquiring, the value for the level of mitapivat, or a pharmaceutical composition thereof, the level of 2,3-DPG, the level of ATP, or the activity of PKR in a subject (e.g., a pediatric patient, an elderly patient or a patient with a disease or condition that makes swallowing difficult) that has been treated with the disclosed mini-tablets, to thereby evaluate the subject. In some aspects, acquiring comprises receiving a sample from the subject. In some aspects, acquiring comprises transmitting the value to another party. In some aspects, the other party is the party that administered the disclosed mini-tablets.

In some aspects, provided herein are methods of treating a subject (e.g., a pediatric patient, an elderly patient or a patient with a disease or condition that makes swallowing difficult), the method comprising: administering to the subject a pharmaceutically effective amount of the disclosed mini-tablets; and acquiring a value for the level of mitapivat, the level of 2,3-diphosphoglycerate (2,3-DPG), the level of adenosine triphosphate (ATP), or the activity of PKR in the subject, to thereby treat the subject.

In other aspects, provided herein are methods for treating thalassemia (e.g., alpha-thalassemia or beta-thalassemia), comprising administering to the subject a pharmaceutically effective amount of the disclosed mini-tablets. Also provided is the disclosed mini-tablet, for use in treating thalassemia (e.g., alpha-thalassemia or beta-thalassemia) in a subject (e.g., a pediatric patient, an elderly patient or a patient with a disease or condition that makes swallowing difficult) in need thereof. Further provided is the use of the disclosed mini-tablet, in the manufacture of a medicament for treating thalassemia (e.g., alpha-thalassemia or beta-thalassemia).

In other aspects, provided herein are methods for increasing the lifetime of red blood cells (RBCs) in a subject (e.g., a pediatric patient, an elderly patient or a patient with a disease or condition that makes swallowing difficult) in need thereof comprising administering to the subject a pharmaceutically effective amount of the disclosed mini-tablet. In one aspect, the disclosed mini-tablet(s) is added directly to whole blood or packed red blood cells extracorporeally. Also provided is the disclosed mini-tablet, for use in increasing the lifetime of red blood cells (RBCs) in a subject (e.g., a pediatric patient, an elderly patient or a patient with a disease or condition that makes swallowing difficult) in need thereof. Further provided is the use of the disclosed mini-tablets, in the manufacture of a medicament for increasing the lifetime of red blood cells (RBCs).

In one aspect, the disclosed methods further comprise the administration or use of the disclosed mini-tablets in combination with folic acid. The administration or use of folic acid can be prior to, during, and/or following the administration or use of the disclosed mini-tablets. In one aspect, however, the folic acid is administered or used prior to and/or concurrently with the disclosed mini-tablets. Thus, in one aspect, provided herein is a method for treating a condition described herein (e.g., PKD, anemia such as hemolytic anemia, acquired hemolytic anemia, thalassemia (e.g., beta-thalassemia, alpha-thalassemia, etc.), sickle cell disease, hereditary spherocytosis, hereditary elliptocytosis, abetalipoproteinemia, Bassen-Kornzweig syndrome, and paroxysmal nocturnal hemoglobinuria); increasing the lifetime of RBCs; regulating 2,3-diphosphoglycerate levels in blood (e.g., in the blood of a subject); activating wild-type or mutant PKR in red blood cells; increasing the amount of hemoglobin; evaluating the level of 2,3-diphosphoglycerate (2,3-DPG), the level of adenosine triphosphate (ATP), or the activity of PKR; evaluating the level of 2,3-diphosphoglycerate (2,3-DPG), the level of adenosine triphosphate (ATP), or the activity of PKR; in a subject (e.g., a pediatric patient, an elderly patient or a patient with a disease or condition that makes swallowing difficult) in need thereof, comprising administering to the subject an effective of the disclosed mini-tablets and folic acid.

In aspects where folic acid is administered or used prior to the disclosed mini-tablets, the folic acid may be used at least 5 days, at least 10 days, at least 15 days, at least 20 days, or at least 25 days prior to the administration or use of the disclosed mini-tablets. In one aspect, the folic acid is administered or used at least 20, at least 21, at least 22, at least 23, at least 24, or at least 25 days prior to the administration or use of the disclosed mini-tablets. In another aspect, the folic acid is administered at least 21 days prior to the administration or the disclosed mini-tablets. In another aspect, the folic acid is administered or used from 1 to 30 days prior to the administration or use of the disclosed mini-tablets. In another aspect, the folic acid is administered or used from 5 to 25 days prior to the administration or use of the disclosed mini-tablets. In another aspect, the folic acid is administered or used from 10 to 30 days prior to the administration or use of the disclosed mini-tablets. In another aspect, the folic acid is administered or used from 10 to 25 days prior to the administration or use of the disclosed mini-tablets. In another aspect, the folic acid is administered or used from 15 to 25 days prior to the administration or use of the disclosed mini-tablets. In another aspect, the folic acid is administered or used from 20 to 25 days prior to the administration or use of the disclosed mini-tablets.

Specific amounts of folic acid to be administered or used with the disclosed mini-tablets will vary depending upon the subject to be treated and the particular mode of administration. In certain aspects, the pharmaceutically effective amount of folic acid is about 0.1 mg to about 10 mg daily. In certain aspects, the effective amount of folic acid is at least 0.1, 0.2, 0.3, 0.4, 0.5, 0.6, 0.7, 0.8, 0.9 or 1.0 mg daily. In one aspect, the effective amount of folic acid is at least 0.8 mg daily or at least 1.0 mg daily.

The amount of folic acid is intended to be combined with any amount of the disclosed mini-tablets. Thus, in certain aspects, provided herein is a method for treating a condition described herein (e.g., PKD, anemia such as hemolytic anemia, acquired hemolytic anemia, thalassemia (e.g., beta-thalassemia, alpha-thalassemiaetc.), sickle cell disease, hereditary spherocytosis, hereditary elliptocytosis, abetalipoproteinemia, Bassen-Kornzweig syndrome, and paroxysmal nocturnal hemoglobinuria); increasing the lifetime of RBCs; regulating 2,3-diphosphoglycerate levels in blood (e.g., in the blood of a subject); activating wild-type or mutant PKR in red blood cells; increasing the amount of hemoglobin; evaluating the level of 2,3-diphosphoglycerate (2,3-DPG), the level of adenosine triphosphate (ATP), or the activity of PKR; evaluating the level of 2,3-diphosphoglycerate (2,3-DPG), the level of adenosine triphosphate (ATP), or the activity of PKR; in a subject (e.g., a pediatric patient, an elderly patient or a patient with a disease or condition that makes swallowing difficult) in need thereof, comprising administering to the subject a pharmaceutically effective amount of a the disclosed mini-tablets and folic acid, wherein the folic acid is administered prior to and/or concurrently with the disclosed mini-tablets (e.g., at least 21 days prior), the disclosed mini-tablets is administered in an amount of 5, 20, or 50 mg BID and wherein the folic acid is administered in an amount of at least 0.8 mg/day.

In certain embodiments, a plurality of the disclosed mini-tablets are provided in an appropriate pharmaceutical dosage system, for example, in a container such as a capsule, a pouch, a sachet or a stick pack. In some embodiments, the pharmaceutical dosage system is a sachet or a stick pack.

### EXEMPLIFICATION

### Example 1 Taste Assessment of Mitapivat in Foods

Mitapivat sulfate tablets were evaluated as three whole and crusted tablets dispersed in food dosing vehicles. Each tablet contained 5.850 mg mitapivat sulfate hydrate (1-(cyclopropylmethyl)-4-(4-(quinoline-8-sulfonamido)benzoyl)piperazin-1-ium hemisulfate sesquihydrate) Form A; 30.09 mg microcrystalline cellulose; 11.56 mg mannitol; 1.500 mg croscarmellose sodium; and 1.000 mg sodium stearyl fumarate with an Opadry® Blue II film coat. The crushed tablets were processed using an automated pill crusher (First Crush, Model: 2002B), as follows: the tablets were inserted into the tablet crusher's cavity, and the lid was closed. The tablet crusher was turned on and the “Extra Grind” pill crushing setting was selected. The tablet powder was transferred to a one-ounce plastic cup and securely closed with a lid to prevent spillage.

Panelists were provided either three (3) AG-348 whole tablets or 3 crushed tablets in a 1-ounce plastic cup, a clean disposable spoon and a 1 ounce plastic sample cup containing 5 mL of the food vehicle. The food vehicles tested were strawberry jam, plain yogurt, strawberry yogurt, applesauce, apple juice, whole milk, chocolate pudding, rice cereal and peanut butter.

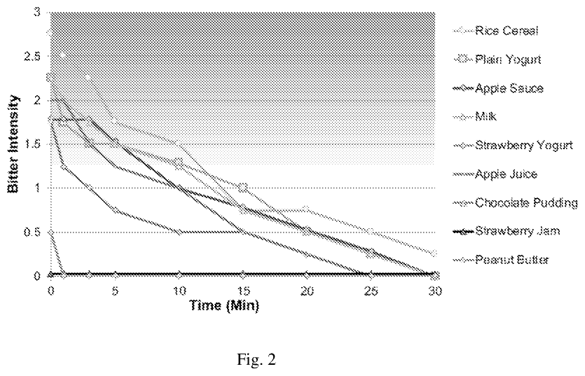

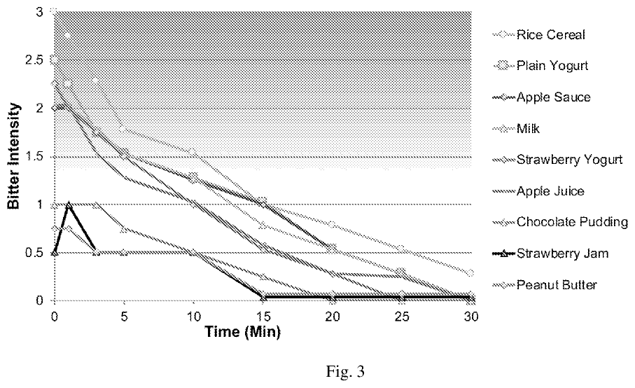

a. Starting at the same time, panelists emptied the whole or crushed tablets into the food vehicle and gently dispersed for 15-30 seconds until homogenous, completely covering the tablets/granules using the spoon. Using a stopwatch, the samples were allowed to sit in the food vehicle for 15 minutes. After 15-minute dwell time, the panelists transferred the food and drug product onto the spoon. At the same time the panelists placed the full sample in their mouth, agitated for 10 seconds and expectorated the mass. The panelists then independently evaluated and recorded the aftertaste characteristics at periodic intervals out to 30 minutes. The sensory panelists evaluated the samples using the Flavor Profile Method of analytical sensory analysis [Keane, P. The Flavor Profile Method, In Hootman (ed.), Manual on Descriptive Analysis Testing for Sensory Evaluation ASTM Manual Series: MNL 13 Baltimore, M D. 1962)] to measure the sensory attributes of products, e.g., basic tastes, aroma, texture and mouthfeel. The Flavor Profile results for bitterness (the primary taste masking challenge of mitapivat sulfate) for the mitapivat sulfate whole and crushed tablets in the nine food dosing vehicles are shown in FIGS. 2 and 3. As expected, the bitterness profiles were higher for the crushed tablets than for whole tablets. Chocolate pudding, strawberry jam and peanut butter show an acceptable profile. Strawberry yogurt and apple sauce are also viable alternatives.

### Example 2 Taste Assessment of Mitapivat Mini Tablets in Foods

Each mitapivat minitablet contained 1.170 mg mitapivat sulfate hydrate (1-(cyclopropylmethyl)-4-(4-(quinoline-8-sulfonamido)benzoyl)piperazin-1-ium hemisulfate sesquihydrate) Form A; 6.020 mg microcrystalline cellulose; 2.310 mg mannitol; 0.300 mg croscarmellose sodium; and 0.200 mg sodium stearyl fumarate with an Opadry® Blue II film coat. The minitablets were manufactured with a direct compression and pan coating process. Multi-tip punches are used in the compression process.

Trained sensory panelists measured the flavor of two doses of mitapivat sulfate minitablets (15 and 30 minitablets) by two methods. Specifically, the granules were evaluated neat and dosed in four food dosing vehicles.

For the neat evaluation, each sensory panelist was provided unit dose of minitablets (either 15 or 30) in sample cup. A stopwatch was started and, panelists placed the pellets on the middle of their tongue. The panelists gently rolled the sample over the tongue continuously until they perceived a 1½-intensity bitterness, recording time to “bitter breakthrough”, and expectorated the disintegrated tablet mass. The mean time to 1½ bitter “bitter breakthrough” was independent of number of minitablets (16-17 seconds).

For the evaluation in Foods: Panelists were provided with mitapivat minitablets (either 15 or 30) in a 1 oz plastic cup, a clean disposable spoon and a loz plastic sample cup containing 5 mL of the food vehicle. Starting at the same time, panelists emptied the whole or crushed minitablets into the food vehicle and gently dispersed for 15-30 seconds until homogenous, completely covering the tablets/granules using the spoon. Using a stopwatch, the samples were allowed to sit in the food vehicle for a predetermined period of time (5, 15, or 30 minutes). After the specified dwell time, the panelists transferred the food and drug product onto the spoon. Starting at the same time the panelists placed the full sample in their mouth, agitated for 10 seconds and expectorated the mass. The panelists then independently evaluated and recorded the aftertaste characteristics at periodic intervals out to 30 minutes. The panelists recited their individual results generating the flavor profile for the sample.

The sensory panelists evaluated the samples using the Flavor Profile Method* of analytical sensory analysis to measure the sensory attributes of products, e.g., basic tastes, aroma, texture and mouthfeel, as described in *Keane, P The Flavor Profile Method. In C. Hootman (Ed.), Manual on Descriptive Analysis Testing for Sensory Evaluation ASTM Manual Series: MNL 13. Baltimore, MD. (1992).

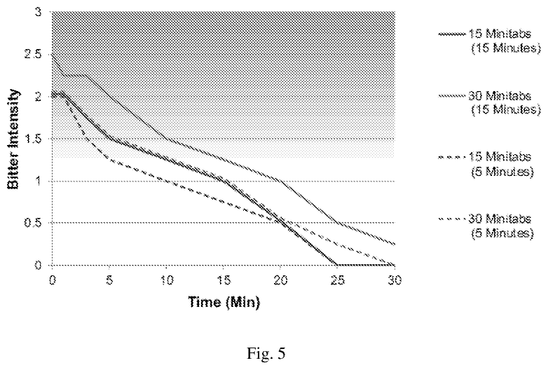

In unsweetened applesauce, 15-minute hold times (both 15 and 30 mini tablets) resulted in bitter profiles above the action threshold (1½), and accordingly were then evaluated at 5 minutes (see FIG. 5).

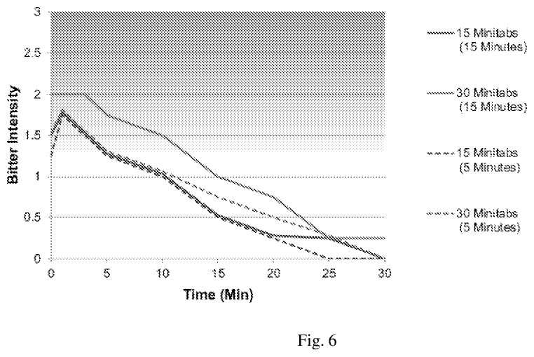

In sweetened applesauce, 15-minute hold times (both 15 and 30 mini tablets) resulted in bitter profiles above the action threshold (1½), and therefore were then evaluated at 5 minutes (see FIG. 6).

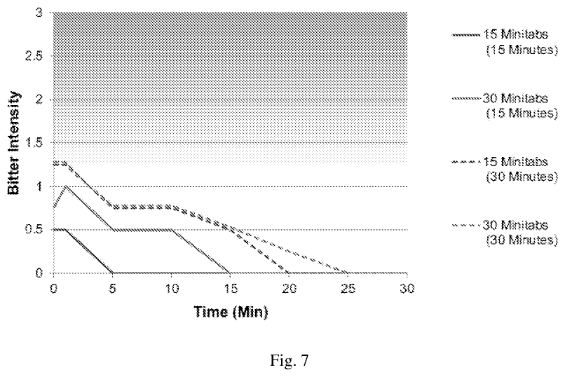

In chocolate pudding, 15-minute hold times (both 15 and 30 mini tablets) resulted in bitter profiles below the action threshold (1½), and therefore were then evaluated at 30 minutes (see FIG. 7).

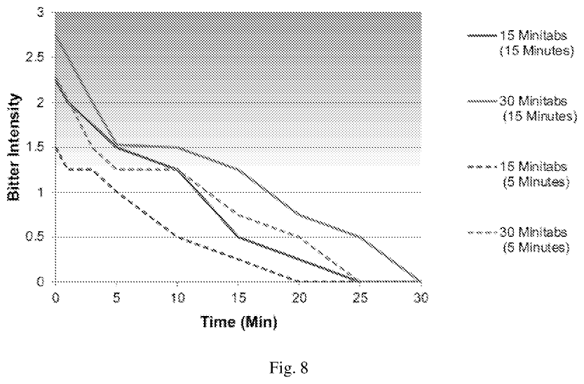

In strawberry yogurt, 15-minute hold times (both 15 and 30 mini tablets) resulted in bitter profiles above the action threshold (1½) and were subsequently evaluated at 5 minutes (see FIG. 8).

Table 1 below shows the initial bitterness score for all vehicles.

| Food Dosing | 15 minitablets | | | 30 minitablets | | |
|---|---|---|---|---|---|---|
| Vehicle | 15 min | 5 min | 30 min | 15 min | 5 min | 30 min |
| Applesauce | 2 | 2 | | 2½ | 2 | |
| (unsweetened) | | | | | | |
| Applesauce | 1½ | ½ | | 2 | 1½ | |
| (sweetened) | | | | | | |
| Chocolate | ½ | | ½ | ½-1 | | ½ |
| Pudding | | | | | | |
| Strawberry | 2-2½ | 1½ | | 2½-3 | −2½ | |
| Yogurt | | | | | | |

Chocolate pudding was found to be a preferred vehicle for the mini tablets. Applesauce (sweetened and/or unsweetened) and strawberry yogurt can also be an acceptable vehicle for the mini tablets.

### Example 3 A Phase 1, Open-Label, Randomized 4-Period Crossover Study to Assess the Relative Bioavailability and Effect of Food on the Coated Granule Formulation of Mitapivat in Healthy Subjects

The primary purpose of this study is to assess the relative bioavailability of the mitapivat coated granule formulation compared to the tablet formulation following a single oral dose of mitapivat under fasted conditions in healthy adult participants. Participants are healthy adults between 18 and 55 years with a body mass index between 18.0 and 32.0 kilograms per square meter (kg/m2). Specifically, the mitapivat tablet will be provided as one mitapivat tablet with 50 mg strength (equivalent to 50 mg of mitapivat). The mitapivat coated granule will be provided as fifty mitapivat granule each with 1 mg strength (equivalent to 1 mg of mitapivat). The formulation of the mitapivat granule is as described in Example 2 above.

Thirty-two eligible subjects will be enrolled to ensure 24 subjects complete the study. Subjects will be randomly assigned to 1 of 4 sequences.

Eight subjects will be randomly assigned to each of the treatment sequences. Each sequence will have 4 periods, in which each subject will receive 1 of the following treatments:
Treatment A: mitapivat tablet (1×50 mg) under fasted conditions
Treatment B: mitapivat coated granules (50×1 mg) under fasted conditions
Treatment C: mitapivat coated granules (50×1 mg) with strawberry yogurt (soft food)
Treatment D: mitapivat coated granules (50×1 mg) with chocolate pudding (soft food).

### Experimental: Treatment Sequence 1: ABCD

Participants will receive Treatment A (mitapivat tablet, orally, under fasted conditions once on Day 1 of Period 1) followed by Treatment B (mitapivat coated granules, orally, under fasted conditions once on Day 1 of Period 2) followed by Treatment C (mitapivat coated granules, with a strawberry yogurt, orally once on Day 1 of Period 3) followed by Treatment D (mitapivat coated granules, with a chocolate pudding, orally once on Day 1 of Period 4). Each Treatment Period will be separated by a Washout Period of 7 days.

### Experimental: Treatment Sequence 2: BDAC

Participants will receive Treatment B (mitapivat coated granules, orally, under fasted conditions once on Day 1 of Period 1) followed by Treatment D (mitapivat coated granules, with a chocolate pudding, orally once on Day 1 of Period 2) followed by Treatment A (mitapivat tablet, orally, under fasted conditions once on Day 1 of Period 3) followed by Treatment C (mitapivat coated granules, with a strawberry yogurt, orally once on Day 1 of Period 4). Each Treatment Period will be separated by a Washout Period of 7 days.

### Experimental: Treatment Sequence 3: CADB

Participants will receive Treatment C (mitapivat coated granules, with a strawberry yogurt, orally once on Day 1 of Period 1) followed by Treatment A (mitapivat tablet, orally, under fasted conditions once on Day 1 of Period 2) followed by Treatment D (mitapivat coated granules, with a chocolate pudding, orally once on Day 1 of Period 3) followed by Treatment B (mitapivat coated granules, orally, under fasted conditions once on Day 1 of Period 4). Each Treatment Period will be separated by a Washout Period of 7 days.

### Experimental: Treatment Sequence 4: DCBA

Participants will receive Treatment D (mitapivat coated granules, with a chocolate pudding, orally once on Day 1 of Period 1) followed by Treatment C (mitapivat coated granules, with a strawberry yogurt, orally once on Day 1 of Period 2) followed by Treatment B (mitapivat coated granules, orally, under fasted conditions once on Day 1 of Period 3) followed by Treatment A (mitapivat tablet, orally, under fasted conditions once on Day 1 of Period 4). Each Treatment Period will be separated by a Washout Period of 7 days.

### Primary Outcome Measure:

1. Maximum Observed Concentration (Cmax) of Mitapivat Under Fasted Conditions
   [Time Frame: Pre-dose and multiple time points post-dose (up to 72 hours)]
2. Area Under the Plasma Concentration-Time Curve from Time Zero to Last Quantifiable Concentration (AUC0-t) of Mitapivat Under Fasted Conditions
   [Time Frame: Pre-dose and multiple time points post-dose (up to 72 hours)]
3. Area Under the Plasma Concentration-Time Curve from Time Zero to Infinity (AUC0-∞) of Mitapivat Under Fasted Conditions
   [Time Frame: Pre-dose and multiple time points post-dose (up to 72 hours)]
4. Time to Reach Maximum Observed Concentration (Tmax) of Mitapivat Under Fasted Conditions

[Time Frame: Pre-dose and multiple time points post-dose (up to 72 hours)]

### Example 4 Pediatric Dosing

The pediatric dosing regimen described herein is weight based (as opposed to age based). The doses per weight group was determined based on a body weight to age curve generated according to the CDC growth chart (source: National Center for Chronic Disease Prevention and Health Promotion, http://www.cdc.gov/growthcharts, and an updated mitapivat adult population PK model which incorporates the effect of allometric scaling of body weight on clearance and volume of distribution. Four different potential body weight and dose cohorts were modeled in order to try to match the typical adult exposure levels of mitapivat at each dose level (5 mg, 20 mg and 50 mg). The four potential weight/dose cohorts were as follows:

| 1 | 2 | 3 | 4 |
|---|---|---|---|
| Less than 20 kg | Less than 20 kg | Less than 20 kg | Less than 20 kg |
| 20 kg to less | 20 kg to less | 20 kg to less | 20 kg to less |
| than 55 kg | than 50 kg | than 45 kg | than 40 kg |
| 55 kg or | 50 kg or | 45 kg or | 40 kg or |
| greater | greater | greater | greater |

Data from the NHANES (National Health and Nutrition Examination Survey) database was then used to obtain a virtual pediatric population to conduct dose simulations that included all PK variables. By comparing the simulated mitapivat exposure ranges (from the 5th to the 95% percentile) from the virtual pediatric population to those in adult subjects, the weight cohorts were subsequently selected as less than 20 kg, 20 kg to less than 40 kg, and 40 kg or greater. From this analysis the following pediatric dose regimen in Table 3 was determined.

TABLE 3

| | | | Recommended | | |
|---|---|---|---|---|---|
| Formu- | Age** | Weight | dosages | | |
| lation | (years) | (kg) | (mg) | (mg) | (mg) |
| minitablet* | 2 to less | greater than or equal to 40 | 50 | 20 | 5 |
| | than 12 | 20 to less than 40 | 20 | 10 | 2 |
| | | less than 20 | 15 | 5 | 1 |
| | 1 to less | | 10 | 4 | 1 |
| | than 2 | | | | |

*minitablet comprises 1 mg of mitapivat per tablet or a pharmaceutically acceptable salt of mitapivat (e.g., mitapivat hemisulfate sesquihydrate) in an amount equivalent to 1 mg of mitapivat with microcrystalline cellulose, croscarmellose sodium, sodium stearyl fumarate, and mannitol.
**If subjects 12 to less than 18 years of age weigh less than 40 kg, dosing by weight as described for 2 to less than 12 years of age should be followed.

### Numbered Embodiments

Embodiment 1a: A method of treating a condition selected from pyruvate kinase deficiency, sickle cell disease, and thalassemia in a pediatric subject comprising administering to the pediatric subject a therapeutically effective amount of mitapivat or a pharmaceutically acceptable salt thereof together with food or water, wherein the pediatric subject is between about 1 year of age to about 12 years of age and has a body weight of about 7 kg to less than about 40 kg.

Embodiment 2a: The method of Embodiment 1a, wherein the pediatric subject is about 2 years of age to less than about 12 years of age.

Embodiment 3a. The method of Embodiment 1a, wherein the pediatric subject is about 1 year of age to less than about 2 years of age.

Embodiment 4a. The method of any one of Embodiments 1a to 3a, wherein the pediatric subject's body weight is about 7 kg to less than about 20 kg.

Embodiment 5a. The method of any one of Embodiments 1a to 3a, wherein the pediatric subject's body weight is about 20 kg to less than about 40 kg.

Embodiment 6a. The method of any one of Embodiments 1a to 5a, wherein the pediatric subject is administered from about 0.5 mg to about 25 mg of mitapivat or an amount of a pharmaceutically acceptable salt of mitapivat that is equivalent to an amount of about 0.5 mg to about 25 mg of mitapivat.

Embodiment 7a. The method of Embodiment 1a, wherein the pediatric subject is administered from about 0.5 mg to about 25 mg of mitapivat or an amount of a pharmaceutically acceptable salt of mitapivat that is equivalent to an amount of about 0.5 mg to about 25 mg of mitapivat; and wherein the pediatric subject is about 2 years of age to less than about 12 years of age and has a body weight of about 20 kg to less than about 40 kg.

Embodiment 8a. The method of Embodiment 1a, wherein the pediatric subject is administered about 15 mg to about 25 mg, about 5 mg to about 15 mg, or about 1 mg to about 5 mg of mitapivat or an amount of a pharmaceutically acceptable salt of mitapivat that is equivalent to an amount of about 15 mg to about 25 mg, about 5 mg to about 15 mg, or about 1 mg to about 5 mg of mitapivat.

Embodiment 9a. The method of Embodiment 8a, wherein the pediatric subject is administered about 20 mg, about 10 mg, or about 2 mg of mitapivat or an amount of a pharmaceutically acceptable salt of mitapivat that is equivalent to about 20 mg, about 10 mg, or about 2 mg of mitapivat.

Embodiment 10a. The method of Embodiment 1a, wherein the pediatric subject is administered about 10 mg to about 20 mg, about 1 mg to about 10 mg, or about 0.5 mg to about 5 mg of mitapivat or an amount of a pharmaceutically acceptable salt of mitapivat that is equivalent to an amount of about 10 mg to about 20 mg, about 1 mg to about 10 mg, or about 0.5 mg to about 5 mg of mitapivat; and wherein the pediatric subject is about 2 years of age to less than about 12 years of age and has a body weight of about 7 kg to less than about 20 kg.

Embodiment 11a. The method of Embodiment 10a, wherein the pediatric subject is administered about 15 mg, about 5 mg, or about 1 mg of mitapivat or an amount of a pharmaceutically acceptable salt of mitapivat that is equivalent to an amount of about 15 mg, about 5 mg, or about 1 mg of mitapivat.

Embodiment 12a. The method of Embodiment 1a, wherein the pediatric subject is administered about 8 mg to about 12 mg, about 2 mg to about 6 mg, or about 0.5 mg to about 3 mg of mitapivat or an amount of a pharmaceutically acceptable salt of mitapivat that is equivalent to an amount of about 8 mg to about 12 mg, about 2 mg to about 6 mg, or about 0.5 mg to about 3 mg of mitapivat; and wherein the pediatric subject is about 1 year of age to less than about 2 years of age.

Embodiment 13a. The method of Embodiment 12a, wherein the pediatric subject is administered about 10 mg, about 4 mg, or about 1 mg of mitapivat or an amount of a pharmaceutically acceptable salt of mitapivat that is equivalent to an amount of about 10 mg, about 4 mg, or about 1 mg of mitapivat.

Embodiment 14a. The method of any one of Embodiments 1a to 13a, wherein the mitapivat or pharmaceutically acceptable salt thereof is administered as part of a pharmaceutical composition comprising one or more granules.

Embodiment 15a. The method of Embodiment 14a, wherein the one or more granules each comprise about 0.5 mg to about 1.5 mg of mitapivat or an amount of a pharmaceutically acceptable salt of mitapivat that is equivalent to an amount of about 0.5 mg to about 1.5 mg of mitapivat.

Embodiment 16a. The method of Embodiment 14a or 15a, wherein the one or more granules each comprise about 1 mg of mitapivat or an amount of a pharmaceutically acceptable salt that is equivalent to an amount of about 1 mg of mitapivat.

Embodiment 17a. The method of any one of Embodiments 1a to 16a, wherein the mitapivat or pharmaceutically acceptable salt thereof is administered once or twice daily.

Embodiment 18a. The method of any one of Embodiments 1a to 17a, wherein the pediatric subject is administered a pharmaceutically acceptable salt of mitapivat.

Embodiment 19a. The method of any one of Embodiments 1a to 18a, wherein the pharmaceutically acceptable salt of mitapivat is a sulfate salt.

Embodiment 20a. The method of any one of Embodiments 1a to 19a, wherein the pharmaceutically acceptable salt of mitapivat is a hemisulfate sesquihydrate salt.

Embodiment 21a. The method of any one of Embodiments 14a to 20a, wherein the one or more granules are administered once or twice daily.

Embodiment 22a. A method of treating a condition selected from pyruvate kinase deficiency, sickle cell disease, and thalassemia in a pediatric subject about 2 years of age to less than about 12 years of age and having a body weight of about 20 kg to less than about 40 kg comprising administering to the pediatric subject one or more granules together with food or water once or twice daily, wherein each granule comprises mitapivat hemisulfate sesquihydrate in an amount equivalent to about 1 mg of mitapivat, and wherein the amount of mitapivat administered to the pediatric subject is about 20 mg, about 10 mg, or about 2 mg.

Embodiment 23a. The method of Embodiment 22a, wherein the amount of mitapivat administered to the pediatric subject is about 20 mg.

Embodiment 24a. The method of Embodiment 22a, wherein the amount of mitapivat administered to the pediatric subject is about 10 mg.

Embodiment 25a. The method of Embodiment 22a, wherein the amount of mitapivat administered to the pediatric subject is about 2 mg.

Embodiment 26a. A method of treating a condition selected from pyruvate kinase deficiency, sickle cell disease, and thalassemia in a pediatric subject about 2 years of age to less than about 12 years of age and having a body weight of less than about 20 kg comprising administering to the pediatric subject one or more granules together with food or water once or twice daily, wherein each granule comprises mitapivat hemisulfate sesquihydrate in an amount equivalent to about 1 mg of mitapivat, and wherein the amount of mitapivat administered to the pediatric subject is about 10 mg, about 5 mg, or about 1 mg.

Embodiment 27a. The method of Embodiment 26a, wherein the amount of mitapivat administered to the pediatric subject is about 10 mg.

Embodiment 28a. The method of Embodiment 26a, wherein the amount of mitapivat administered to the pediatric subject is about 5 mg.

Embodiment 29a. The method of Embodiment 26a, wherein the amount of mitapivat administered to the pediatric subject is about 1 mg.

Embodiment 30a. A method of treating a condition selected from pyruvate kinase deficiency, sickle cell disease, and thalassemia in a pediatric subject about 1 year of age to less than about 2 years of age and having a body weight of about 7 kg to less than about 20 kg comprising administering to the pediatric subject one or more granules together with food or water once or twice daily, wherein each granule comprises mitapivat hemisulfate sesquihydrate in an amount equivalent to about 1 mg of mitapivat, and wherein the amount of mitapivat administered to the subject is about 10 mg, about 4 mg, or about 1 mg.

Embodiment 31a. The method of Embodiment 30a, wherein the amount of mitapivat administered to the pediatric subject is about 10 mg.

Embodiment 32a. The method of Embodiment 30a, wherein the amount of mitapivat administered to the pediatric subject is about 4 mg.

Embodiment 33a. The method of Embodiment 30a, wherein the amount of mitapivat administered to the pediatric subject is about 1 mg.

Embodiment 34a. The method of any one of Embodiments 15a to 32a, wherein the one or more granules further comprise one or more inactive ingredients.

Embodiment 35a. The method of Embodiment 34a, wherein the one or more inactive ingredients are selected from a disintegrant, lubricant, binder, and diluent.

Embodiment 36a. The method of Embodiment 34a or 35a, wherein the one or more inactive ingredients are selected from microcrystalline cellulose, mannitol, croscarmellose sodium and sodium stearyl fumarate.

Embodiment 37a. The method of any one of Embodiments 15a to 36a, wherein the one or more granules further comprise a film-coat.

Embodiment 38a. A method of treating a condition selected from pyruvate kinase deficiency, sickle cell disease, and thalassemia in a pediatric subject between about 1 year of age to about 12 years of age and having a body weight of about 7 kg to less than about 40 kg comprising administering to the pediatric subject an initial amount of mitapivat or a pharmaceutically acceptable salt thereof with food or water for a first period of time; assessing the pediatric subject's hemoglobin levels after the first period of time to determine whether the pediatric subject is within a target hemoglobin level; and continuing to administer the initial amount of mitapivat or a pharmaceutically acceptable salt thereof with food or water if the pediatric subject's hemoglobin level is within the target hemoglobin level or administering a first adjusted amount of mitapivat or a pharmaceutically acceptable salt thereof with food or water if the pediatric subject's hemoglobin level is not within the target hemoglobin level.

Embodiment 39a. The method of Embodiment 38a, wherein the method further comprises administering the first adjusted amount of mitapivat or a pharmaceutically acceptable salt thereof with food or water for a second period of time; assessing the pediatric subject's hemoglobin levels after the second period of time to determine whether the pediatric subject is within the target hemoglobin level; and continuing to administer the first adjusted amount if the pediatric subject's hemoglobin level is within the target hemoglobin level or administering a second adjusted amount of mitapivat or a pharmaceutically acceptable salt thereof with food or water, if the pediatric subject's hemoglobin level is not within the target hemoglobin level after the second period time.

Embodiment 40a. The method of Embodiment 38a or 39a, wherein the method further comprises administering a third adjusted amount of mitapivat or a pharmaceutically acceptable salt thereof with food or water for a third period of time if the pediatric subject's hemoglobin level is not within the target hemoglobin level after the second period time; or continuing to administer the second adjusted amount if the pediatric subject's hemoglobin level is within the target hemoglobin level.

Embodiment 41a. The method of any one of Embodiments 38a to 40a, wherein the method further comprises continuing to assess the pediatric subject's hemoglobin levels and re-adjusting the amount of mitapivat or a pharmaceutically acceptable salt thereof until the pediatric subject's hemoglobin level is within the target hemoglobin level, or continuing to administer mitapivat or a pharmaceutically acceptable salt thereof to the pediatric subject without further adjustment.

Embodiment 42a. The method of any one of Embodiments 38a to 41a, wherein the pediatric subject is about 2 years of age to less than about 12 years of age.

Embodiment 43a. The method of any one of Embodiments 38a to 42a, wherein the pediatric subject is about 1 year of age to less than about 2 years of age.

Embodiment 44a. The method of any one of Embodiments 38a to 43a, wherein the pediatric subject's body weight is about 7 kg to less than about 20 kg.

Embodiment 45a. The method of any one of Embodiments 38a to 44a, wherein the pediatric subject's body weight is about 20 kg to less than about 40 kg.

Embodiment 46a. The method of any one of Embodiments 38a to 45a, wherein the initial amount of mitapivat is about 0.5 mg to about 25 mg of mitapivat, or wherein the initial amount of the pharmaceutically acceptable salt of mitapivat is an amount that is equivalent to about 0.5 mg to about 25 mg of mitapivat.

Embodiment 47a. The method of any one of Embodiments 38a to 46a, wherein the initial amount of mitapivat is about 15 mg to about 25 mg, about 5 mg to about 15 mg, or about 1 mg to about 5 mg of mitapivat, or wherein the initial amount of the pharmaceutically acceptable salt of mitapivat is an amount that is equivalent to about 15 mg to about 25 mg, about 5 mg to about 15 mg, or about 1 mg to about 5 mg of mitapivat, and wherein the pediatric subject is about 2 years of age to less than about 12 years of age and has a body weight of about 20 kg to less than about 40 kg.

Embodiment 48a. The method of Embodiment 47a, wherein the initial amount of mitapivat is about 20 mg, about 10 mg, or about 2 mg mitapivat, or wherein the initial amount of the pharmaceutically acceptable salt of mitapivat is an amount that is equivalent to about 20 mg, about 10 mg, or about 2 mg of mitapivat.

Embodiment 49a. The method of any one of Embodiments 38a to 41a, wherein the initial amount of mitapivat is about 10 mg to about 20 mg, about 1 mg to about 10 mg, or about 0.5 mg to about 5 mg of mitapivat, or wherein the initial amount of the pharmaceutically acceptable salt of mitapivat is an amount that is equivalent to about 10 mg to about 20 mg, about 1 mg to about 10 mg, or about 0.5 mg to about 5 mg of mitapivat, and wherein the pediatric subject is about 2 years of age to less than about 12 years of age and has a body weight of about 7 kg to about less than 20 kg.

Embodiment 50a. The method of Embodiment 49a, wherein the initial amount of mitapivat is about 15 mg, about 5 mg, or about 1 mg of mitapivat, or wherein the initial amount of the pharmaceutically acceptable salt of mitapivat is an amount that is equivalent to about 15 mg, about 5 mg, or about 1 mg of mitapivat.

Embodiment 51a. The method of any one of Embodiments 38a to 41a, wherein the initial amount of mitapivat is about 8 mg to about 12 mg, about 2 mg to about 6 mg, or about 0.5 mg to about 3 mg of mitapivat, or wherein the initial amount of the pharmaceutically acceptable salt of mitapivat is an amount that is equivalent to about 8 mg to about 12 mg, about 2 mg to about 6 mg, or about 0.5 mg to about 3 mg of mitapivat, and wherein the pediatric subject is about 1 year of age to less than about 2 years of age.

Embodiment 52a. The method of Embodiment 51a, wherein the initial amount of mitapivat is about 10 mg, about 4 mg, or about 1 mg of mitapivat, or wherein the initial amount of the pharmaceutically acceptable salt of mitapivat is an amount that is equivalent to about 10 mg, about 4 mg, or about 1 mg of mitapivat.

Embodiment 53a. The method of any one of Embodiments 1a to 52a, wherein the condition is pyruvate kinase deficiency.

Embodiment 54a. The method of any one of Embodiments 1a to 52a, wherein the condition is sickle cell disease.

Embodiment 55a. The method of any one of Embodiments 1a to 52a, wherein the condition is thalassemia.

Embodiment 55b. The method of any one of Embodiments 1a to 52a, further comprising administering folic acid to the subject.

Embodiment 56a. A pharmaceutical minitablet comprising about 0.1 mg to about 5 mg of mitapivat or a pharmaceutically acceptable salt thereof in an amount that is equivalent to about 0.1 mg to about 5 mg of mitapivat and having as its longest dimension or diameter a length of about 10.0 mm to about 0.1 mm, wherein the minitablet is suitable for mixing with food before oral administration once or twice daily to patients with difficulties swallowing.

Embodiment 57a. The minitablet of Embodiment 56a, wherein the patients are between about 1 year to about 12 years of age and weighing between about 7 kg to less than about 40 kg.

Embodiment 58a. The minitablet of Embodiment 56a, wherein the patients are elderly.

Embodiment 59a. A pharmaceutical minitablet comprising about 0.1 mg to about 5 mg of mitapivat or a pharmaceutically acceptable salt thereof in an amount that is equivalent to about 0.1 mg to about 5 mg of mitapivat and having as its longest dimension or diameter a length of about 10.0 mm to about 0.1 mm for oral administration once or twice daily to patients that are between about 1 year to about 12 years of age and weighing between about 7 kg to less than about 40 kg or adult patients with difficulties swallowing.

Embodiment 60a. The minitablet of Embodiment 59a, wherein the minitablet is suitable for mixing with food before administration.

Embodiment 61a. The minitablet of any one of Embodiments 56a to 60a, wherein the minitablet comprises about 1 mg of mitapivat or a pharmaceutically acceptable salt thereof in an amount that is equivalent to about 1 mg of mitapivat.

Embodiment 62a. The minitablet of any one of Embodiments 56a to 61a wherein the minitablet further comprises at least one of microcrystalline cellulose, mannitol, croscarmellose sodium and sodium stearyl fumarate.

Embodiment 63a. A method of treating a condition selected from pyruvate kinase deficiency, sickle cell disease, and thalassemia in a pediatric subject about 1 year of age to about 12 years of age comprising administering to the pediatric subject an initial amount of mitapivat or a pharmaceutically acceptable salt thereof, once or twice daily, for a first period of time, wherein the initial amount administered is: ii) about 0.5 mg to about 25 mg of mitapivat or an amount of a pharmaceutically salt of mitapivat that is equivalent to about 0.5 mg to about 25 mg of mitapivat if the pediatric subject's body weight is about 7 kg to less than about 40 kg; iv) about 0.5 mg to about 25 mg of mitapivat or an amount of a pharmaceutically acceptable salt that is equivalent to about 0.5 mg to about 25 mg of mitapivat if the pediatric subject's body weight is about 7 kg to less than about 20 kg; or v) about 0.5 mg to about 15 mg of mitapivat or an amount of a pharmaceutically acceptable salt that is equivalent to about 0.5 mg to about 15 mg of mitapivat if the pediatric subject is about 1 year of age to less than about 2 years of age and the pediatric subject's body weight is at least 7 kg; assessing the body weight of the pediatric subject over a period of time; and continuing to administer the initial amount of mitapivat or a pharmaceutically acceptable salt thereof if the pediatric subject's body weight remains within the same range, or administering a first adjusted amount of mitapivat or a pharmaceutically acceptable salt thereof based on the pediatric subject's weight falling within the ranges listed in i)-v) above.

Embodiment 54b. A method of reducing an established daily amount of mitapivat or discontinuing treatment of mitapivat or a pharmaceutically acceptable salt thereof for a pediatric subject between about 1 year of age to about 12 years of age and having a body weight of about 7 kg to less than about 40 kg who is being administered an established daily amount of mitapivat or a pharmaceutically acceptable salt thereof with food or water, the method comprising a) administering to the pediatric subject a first adjusted amount of mitapivat or pharmaceutically acceptable salt thereof with food or water for a first period of time, wherein the first adjusted amount is less than the established daily amount; and b) monitoring the pediatric subject for acute hemolysis or anemia, or both, during the first period of time.

Embodiment 55c. The method of Embodiment 54b, comprising repeating step a) and step b) using a second adjusted amount of mitapivat or a pharmaceutically acceptable salt thereof together with food or water for a second period of time and continuing to monitor the pediatric subject for symptoms of acute hemolysis or anemia or both, wherein the second adjusted amount is less than the first adjusted amount.

Embodiment 56b. The method of Embodiment 54b or 55c, further comprising the step of treating the pediatric subject for acute hemolysis or anemia, or both, if the subject shows symptoms of acute hemolysis or anemia, or both.

Embodiment 57b. The method of any one of Embodiments 54b to 56b, wherein administration of the second adjusted amount of mitapivat or the pharmaceutically acceptable salt thereof is initiated immediately after completion of the first period of time.

Embodiment 58b. The method of any one of Embodiments 54b to 57b, further comprising repeating step a) and step b) using a third adjusted amount of mitapivat or a pharmaceutically acceptable salt thereof together with food or water for a third period of time and continuing to monitor the pediatric subject for symptoms of acute hemolysis or anemia or both, wherein the third adjusted amount is less than the second adjusted amount administered in the previous step a).

Embodiment 59b. The method of Embodiment 58b, wherein administration of the third adjusted amount of mitapivat or the pharmaceutically acceptable salt thereof is initiated immediately after completion of the second period of time.

Embodiment 60b. The method of any one of Embodiments 54b to 59b, wherein step a) and b) are repeated until the pediatric subject is no longer being administered mitapivat or a pharmaceutically acceptable salt thereof.

Embodiment 61b. The method of any one of Embodiments 54b to 60b, further comprising the step of treating the pediatric subject for acute hemolysis or anemia, if the pediatric subject shows symptoms of acute hemolysis or anemia.

Embodiment 62b. The method of Embodiment 61b, wherein treating is continued until the symptoms of acute hemolysis or anemia, or both improve.

Embodiment 63b. The method of any one of Embodiments 55b to 62b, wherein the first adjusted amount of mitapivat or the pharmaceutically acceptable salt is about 50% to about 75% less than the established daily amount of mitapivat or a pharmaceutically acceptable salt thereof and is administered once a day (QD).

Embodiment 64b. The method of any one of Embodiments 55b to 63b, wherein the second adjusted amount of mitapivat or the pharmaceutically acceptable salt is about 50% to about 70% less than the first adjusted amount of mitapivat or the pharmaceutically acceptable salt and the subject is administered the second adjusted amount once every day (QD).

Embodiment 65b. The method of any one of Embodiments 58b to 63b, wherein the third adjusted amount of mitapivat or the pharmaceutically acceptable salt is equivalent to the amount of the second adjusted amount of mitapivat or the pharmaceutically acceptable salt administered every other day (QOD).

Embodiment 66b. The method of any one of Embodiments 54b to 65b, wherein acute hemolysis is characterized by a rapid loss in hemoglobin.

Embodiment 67b. The method of any one of Embodiments 36b to 66b, wherein the subject is administered a pharmaceutically acceptable salt of mitapivat.

Embodiment 68b. The method of any one of Embodiments 36b to 67b, wherein the pharmaceutically acceptable salt is a sulfate salt.

Embodiment 69b. The method of any one of Embodiments 36b to 68b, wherein the pharmaceutically acceptable salt is a hemisulfate sesquihydrate salt.

Embodiment 70b. The method of any one of Embodiments 51b to 69b, wherein the subject is being treated for a condition selected from pyruvate kinase deficiency (PKD), thalassemia, and sickle cell disease (SCD).

Embodiment 71b. The method of any one of Embodiments 51b to 70b, wherein the subject is being treated for pyruvate kinase deficiency (PKD).

Embodiment 72b. The method of any one of Embodiments 51b to 70b, wherein the subject is being treated for sickle cell disease (SCD).

Embodiment 73b. The method of any one of Embodiments 51b to 70b, wherein the subject is being treated for thalassemia.

Embodiment 62d. The method of any one of the preceding numbered embodiments having a first, second, and/or third period of time, wherein the first, second, and third period of time are each independently selected from about 1 to about 12 weeks, about 1 to about 11 weeks, about 1 to about 10 weeks, about 1 to about 9 weeks, about 1 to about 8 weeks, about 1 to about 7 weeks, about 1 to about 6 weeks, about 1 to about 5 weeks, about 1 to about 4 weeks, about 1 to about 3 weeks, about 1 to about 2 weeks, about 2 to about 12 weeks, about 2 to about 11 weeks, about 2 to about 10 weeks, about 2 to about 9 weeks, about 2 to about 8 weeks, about 2 to about 7 weeks, about 2 to about 6 weeks, about 2 to about 5 weeks, about 2 to about 4 weeks, about 2 to about 3 weeks, about 3 to about 12 weeks, about 3 to about 11 weeks, about 3 to about 10 weeks, about 3 to about 9 weeks, about 3 to about 8 weeks, about 3 to about 7 weeks, about 3 to about 6 weeks, about 3 to about 5 weeks, about 3 to about 4 weeks, about 4 to about 12 weeks, about 4 to about 11 weeks, about 4 to about 10 weeks, about 4 to about 9 weeks, about 4 to about 8 weeks, about 4 to about 7 weeks, about 4 to about 6 weeks, about 4 to about 5 weeks, about 5 to about 12 weeks, about 5 to about 11 weeks, about 5 to about 10 weeks, about 5 to about 9 weeks, about 5 to about 8 weeks, about 5 to about 7 weeks, about 5 to about 6 weeks, about 6 to about 12 weeks, about 6 to about 11 weeks, about 6 to about 10 weeks, about 6 to about 9 weeks, about 6 to about 8 weeks, and about 6 to about 7 weeks.

Embodiment 63c. The method of any one of the preceding numbered embodiments having a first, second, and/or third period of time, wherein the first, second, and third period of time are each independently selected from at least about 1 week, at least about 2 weeks, at least about 3 three weeks, at least about 4 weeks, at least about 5 weeks, at least about 6 weeks, at least about 7 weeks, at least about 8 weeks, at least about 9 weeks, at least about 10 weeks, at least about 11 weeks, and at least about 12 weeks.

Embodiment 64c. The method of any one of the preceding numbered embodiments having a first, second, and/or third period of time, wherein the first, second, and third period of time are each independently selected from at most about 1 week, at most about 2 weeks, at most about 3 weeks, at most about 4 weeks, at most about 5 weeks, at most about 6 weeks, at most about 7 weeks, at most about 8 weeks, at most about 9 weeks, at most about 10 weeks, at most about 11 weeks, and at most about 12 weeks.

Embodiment 65c. The method of any one of the preceding numbered embodiments having a first, second, and/or third period of time, wherein the first, second, and third period of time are each independently selected from about 1 week, about 2 weeks, about 3 weeks, about 4 weeks, about 5 weeks, about 6 weeks, about 7 weeks, about 8 weeks, about 9 weeks, about 10 weeks, about 11 weeks and about 12 weeks.

Embodiment 66c. The method of any one of the preceding numbered embodiments having a first, second, and/or third period of time, wherein the first and second period of time are the same duration; the first and third period of time are the same duration; the second and third period of time are the same duration; or the first, second, and third period of time are all the same duration.

Embodiment 67d. A method of reducing an established daily amount of mitapivat or discontinuing treatment of mitapivat or a pharmaceutically acceptable salt thereof for a pediatric subject between about 1 year of age to about 12 years of age and having a body weight of about 7 kg to less than about 40 kg who is being administered an established daily amount of mitapivat or a pharmaceutically acceptable salt thereof, the method comprising immediately discontinuing administration of mitapivat or the pharmaceutically acceptable salt thereof, wherein the pediatric subject has experienced an adverse event.

Embodiment 68d. The method of Embodiment 67d further comprising monitoring the pediatric subject for symptoms of acute hemolysis or anemia or both.

In some aspects of any of the foregoing embodiments or description, the administration of mitapivat, or a pharmaceutically acceptable salt thereof, is immediately ceased (e.g., in the event of the subject suffering an adverse event) and the subject is monitored for acute hemolysis or anemia, or both, during a period of time.

While a number of embodiments have been described, the scope of this disclosure is to be defined by the appended claims, and not by the specific embodiments that have been represented by way of example. The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating a condition selected from pyruvate kinase deficiency, sickle cell disease, and thalassemia in a pediatric subject comprising administering to the pediatric subject a therapeutically effective amount of mitapivat or a pharmaceutically acceptable salt thereof together with food or water, wherein the pediatric subject is between about 1 year of age to about 12 years of age and has a body weight of about 7 kg to less than about 40 kg.

2. The method of claim 1, wherein the pediatric subject is about 2 years of age to less than about 12 years of age.

3. The method of claim 1, wherein the pediatric subject is about 1 year of age to less than about 2 years of age.

4. The method of any one of claims 1 to 3, wherein the pediatric subject's body weight is about 7 kg to less than about 20 kg.

5. The method of any one of claims 1 to 3, wherein the pediatric subject's body weight is about 20 kg to less than about 40 kg.

6. The method of any one of claims 1 to 5, wherein the pediatric subject is administered from about 0.5 mg to about 25 mg of mitapivat or an amount of a pharmaceutically acceptable salt of mitapivat that is equivalent to an amount of about 0.5 mg to about 25 mg of mitapivat.

7. The method of claim 1, wherein the pediatric subject is administered from about 0.5 mg to about 25 mg of mitapivat or an amount of a pharmaceutically acceptable salt of mitapivat that is equivalent to an amount of about 0.5 mg to about 25 mg of mitapivat; and wherein the pediatric subject is about 2 years of age to less than about 12 years of age and has a body weight of about 20 kg to less than about 40 kg.

8. The method of claim 1, wherein the pediatric subject is administered about 15 mg to about 25 mg, about 5 mg to about 15 mg, or about 1 mg to about 5 mg of mitapivat or an amount of a pharmaceutically acceptable salt of mitapivat that is equivalent to an amount of about 15 mg to about 25 mg, about 5 mg to about 15 mg, or about 1 mg to about 5 mg of mitapivat.

9. The method of claim 8, wherein the pediatric subject is administered about 20 mg, about 10 mg, or about 2 mg of mitapivat or an amount of a pharmaceutically acceptable salt of mitapivat that is equivalent to about 20 mg, about 10 mg, or about 2 mg of mitapivat.

10. The method of claim 1, wherein the pediatric subject is administered about 10 mg to about 20 mg, about 1 mg to about 10 mg, or about 0.5 mg to about 5 mg of mitapivat or an amount of a pharmaceutically acceptable salt of mitapivat that is equivalent to an amount of about 10 mg to about 20 mg, about 1 mg to about 10 mg, or about 0.5 mg to about 5 mg of mitapivat; and wherein the pediatric subject is about 2 years of age to less than about 12 years of age and has a body weight of about 7 kg to less than about 20 kg.

11. The method of claim 10, wherein the pediatric subject is administered about 15 mg, about 5 mg, or about 1 mg of mitapivat or an amount of a pharmaceutically acceptable salt of mitapivat that is equivalent to an amount of about 15 mg, about 5 mg, or about 1 mg of mitapivat.

12. The method of claim 1, wherein the pediatric subject is administered about 8 mg to about 12 mg, about 2 mg to about 6 mg, or about 0.5 mg to about 3 mg of mitapivat or an amount of a pharmaceutically acceptable salt of mitapivat that is equivalent to an amount of about 8 mg to about 12 mg, about 2 mg to about 6 mg, or about 0.5 mg to about 3 mg of mitapivat; and wherein the pediatric subject is about 1 year of age to less than about 2 years of age.

13. The method of claim 12, wherein the pediatric subject is administered about 10 mg, about 4 mg, or about 1 mg of mitapivat or an amount of a pharmaceutically acceptable salt of mitapivat that is equivalent to an amount of about 10 mg, about 4 mg, or about 1 mg of mitapivat.

14. The method of any one of claims 1 to 13, wherein the mitapivat or pharmaceutically acceptable salt thereof is administered as part of a pharmaceutical composition comprising one or more granules.

15. The method of claim 14, wherein the one or more granules each comprise about 0.5 mg to about 1.5 mg of mitapivat or an amount of a pharmaceutically acceptable salt of mitapivat that is equivalent to about 0.5 mg to about 1.5 mg of mitapivat.

16. The method of claim 14 or 15, wherein the one or more granules each comprise about 1 mg of mitapivat or an amount of a pharmaceutically acceptable salt that is equivalent to about 1 mg of mitapivat.

17. The method of any one of claims 1 to 16, wherein the mitapivat or pharmaceutically acceptable salt thereof is administered once or twice daily.

18. The method of any one of claims 1 to 17, wherein the pediatric subject is administered a pharmaceutically acceptable salt of mitapivat.

19. The method of any one of claims 1 to 18, wherein the pharmaceutically acceptable salt of mitapivat is a sulfate salt.

20. The method of any one of claims 1 to 19, wherein the pharmaceutically acceptable salt of mitapivat is a hemisulfate sesquihydrate salt.

21. The method of any one of claims 14 to 20, wherein the one or more granules are administered once or twice daily.

22. A method of treating a condition selected from pyruvate kinase deficiency, sickle cell disease, and thalassemia in a pediatric subject about 2 years of age to less than about 12 years of age and having a body weight of about 20 kg to less than about 40 kg comprising administering to the pediatric subject one or more granules together with food or water once or twice daily, wherein each granule comprises mitapivat hemisulfate sesquihydrate in an amount equivalent to about 1 mg of mitapivat, and wherein the amount of mitapivat administered to the pediatric subject is about 20 mg, about 10 mg, or about 2 mg.

23. The method of claim 22, wherein the amount of mitapivat administered to the pediatric subject is about 20 mg.

24. The method of claim 22, wherein the amount of mitapivat administered to the pediatric subject is about 10 mg.

25. The method of claim 22, wherein the amount of mitapivat administered to the pediatric subject is about 2 mg.

26. A method of treating a condition selected from pyruvate kinase deficiency, sickle cell disease, and thalassemia in a pediatric subject about 2 years of age to less than about 12 years of age and having a body weight of less than about 20 kg comprising administering to the pediatric subject one or more granules together with food or water once or twice daily, wherein each granule comprises mitapivat hemisulfate sesquihydrate in an amount equivalent to about 1 mg of mitapivat, and wherein the amount of mitapivat administered to the pediatric subject is about 10 mg, about 5 mg, or about 1 mg.

27. The method of claim 26, wherein the amount of mitapivat administered to the pediatric subject is about 10 mg.

28. The method of claim 26, wherein the amount of mitapivat administered to the pediatric subject is about 5 mg.

29. The method of claim 26, wherein the amount of mitapivat administered to the pediatric subject is about 1 mg.

30. A method of treating a condition selected from pyruvate kinase deficiency, sickle cell disease, and thalassemia in a pediatric subject about 1 year of age to less than about 2 years of age and having a body weight of about 7 kg to less than about 20 kg comprising administering to the pediatric subject one or more granules together with food or water once or twice daily, wherein each granule comprises mitapivat hemisulfate sesquihydrate in an amount equivalent to about 1 mg of mitapivat, and wherein the amount of mitapivat administered to the subject is about 10 mg, about 4 mg, or about 1 mg.

31. The method of claim 30, wherein the amount of mitapivat administered to the pediatric subject is about 10 mg.

32. The method of claim 30, wherein the amount of mitapivat administered to the pediatric subject is about 4 mg.

33. The method of claim 30, wherein the amount of mitapivat administered to the pediatric subject is about 1 mg.

34. The method of any one of claims 15 to 32, wherein the one or more granules further comprise one or more inactive ingredients.

35. The method of claim 34, wherein the one or more inactive ingredients are selected from a disintegrant, lubricant, binder, and diluent.

36. The method of claim 34 or 35, wherein the one or more inactive ingredients are selected from microcrystalline cellulose, mannitol, croscarmellose sodium and sodium stearyl fumarate.

37. The method of any one of claims 15 to 36, wherein the one or more granules further comprise a film-coat.

38. A method of treating a condition selected from pyruvate kinase deficiency, sickle cell disease, and thalassemia in a pediatric subject between about 1 year of age to about 12 years of age and having a body weight of about 7 kg to less than about 40 kg comprising
administering to the pediatric subject an initial amount of mitapivat or a pharmaceutically acceptable salt thereof with food or water for a first period of time;
assessing the pediatric subject's hemoglobin levels after the first period of time to determine whether the pediatric subject is within a target hemoglobin level; and
continuing to administer the initial amount of mitapivat or a pharmaceutically acceptable salt thereof with food or water if the pediatric subject's hemoglobin level is within the target hemoglobin level or administering a first adjusted amount of mitapivat or a pharmaceutically acceptable salt thereof with food or water if the pediatric subject's hemoglobin level is not within the target hemoglobin level.

39. The method of claim 38, wherein the method further comprises administering the first adjusted amount of mitapivat or a pharmaceutically acceptable salt thereof with food or water for a second period of time;
assessing the pediatric subject's hemoglobin levels after the second period of time to determine whether the pediatric subject is within the target hemoglobin level; and
continuing to administer the first adjusted amount if the pediatric subject's hemoglobin level is within the target hemoglobin level or administering a second adjusted amount of mitapivat or a pharmaceutically acceptable salt thereof with food or water, if the pediatric subject's hemoglobin level is not within the target hemoglobin level after the second period time.

40. The method of claim 38 or 39, wherein the method further comprises administering a third adjusted amount of mitapivat or a pharmaceutically acceptable salt thereof with food or water for a third period of time if the pediatric subject's hemoglobin level is not within the target hemoglobin level after the second period time; or continuing to administer the second adjusted amount if the pediatric subject's hemoglobin level is within the target hemoglobin level.

41. The method of any one of claims 38 to 40, wherein the method further comprises continuing to assess the pediatric subject's hemoglobin levels and re-adjusting the amount of mitapivat or a pharmaceutically acceptable salt thereof until the pediatric subject's hemoglobin level is within the target hemoglobin level, or continuing to administer mitapivat or a pharmaceutically acceptable salt thereof to the pediatric subject without further adjustment.

42. The method of any one of claims 38 to 41, wherein the pediatric subject is about 2 years of age to less than about 12 years of age.

43. The method of any one of claims 38 to 42, wherein the pediatric subject is about 1 year of age to less than about 2 years of age.

44. The method of any one of claims 38 to 43, wherein the pediatric subject's body weight is about 7 kg to less than about 20 kg.

45. The method of any one of claims 38 to 44, wherein the pediatric subject's body weight is about 20 kg to less than about 40 kg.

46. The method of any one of claims 38 to 45, wherein the initial amount of mitapivat is about 0.5 mg to about 25 mg of mitapivat, or wherein the initial amount of the pharmaceutically acceptable salt of mitapivat is an amount that is equivalent to about 0.5 mg to about 25 mg of mitapivat.

47. The method of any one of claims 38 to 46, wherein the initial amount of mitapivat is about 15 mg to about 25 mg, about 5 mg to about 15 mg, or about 1 mg to about 5 mg of mitapivat, or wherein the initial amount of the pharmaceutically acceptable salt of mitapivat is an amount that is equivalent to about 15 mg to about 25 mg, about 5 mg to about 15 mg, or about 1 mg to about 5 mg of mitapivat, and wherein the pediatric subject is about 2 years of age to less than about 12 years of age and has a body weight of about 20 kg to less than about 40 kg.

48. The method of claim 47, wherein the initial amount of mitapivat is about 20 mg, about 10 mg, or about 2 mg mitapivat, or wherein the initial amount of the pharmaceutically acceptable salt of mitapivat is an amount that is equivalent to about 20 mg, about 10 mg, or about 2 mg of mitapivat.

49. The method of any one of claims 38 to 41, wherein the initial amount of mitapivat is about 10 mg to about 20 mg, about 1 mg to about 10 mg, or about 0.5 mg to about 5 mg of mitapivat, or wherein the initial amount of the pharmaceutically acceptable salt of mitapivat is an amount that is equivalent to about 10 mg to about 20 mg, about 1 mg to about 10 mg, or about 0.5 mg to about 5 mg of mitapivat, and wherein the pediatric subject is about 2 years of age to less than about 12 years of age and has a body weight of about 7 kg to about less than 20 kg.

50. The method of claim 49, wherein the initial amount of mitapivat is about 15 mg, about 5 mg, or about 1 mg of mitapivat, or wherein the initial amount of the pharmaceutically acceptable salt of mitapivat is an amount that is equivalent to about 15 mg, about 5 mg, or about 1 mg of mitapivat.

51. The method of any one of claims 38 to 41, wherein the initial amount of mitapivat is about 8 mg to about 12 mg, about 2 mg to about 6 mg, or about 0.5 mg to about 3 mg of mitapivat, or wherein the initial amount of the pharmaceutically acceptable salt of mitapivat is an amount that is equivalent to about 8 mg to about 12 mg, about 2 mg to about 6 mg, or about 0.5 mg to about 3 mg of mitapivat, and wherein the pediatric subject is about 1 year of age to less than about 2 years of age.

52. The method of claim 51, wherein the initial amount of mitapivat is about 10 mg, about 4 mg, or about 1 mg of mitapivat, or wherein the initial amount of the pharmaceutically acceptable salt of mitapivat is an amount that is equivalent to about 10 mg, about 4 mg, or about 1 mg of mitapivat.

53. The method of any one of claims 1 to 52, wherein the condition is pyruvate kinase deficiency.

54. The method of any one of claims 1 to 52, wherein the condition is sickle cell disease.

55. The method of any one of claims 1 to 52, wherein the condition is thalassemia.

56. A pharmaceutical minitablet comprising about 0.1 mg to about 5 mg of mitapivat or a pharmaceutically acceptable salt thereof in an amount that is equivalent to about 0.1 mg to about 5 mg of mitapivat and having as its longest dimension or diameter a length of about 10.0 mm to about 0.1 mm, wherein the minitablet is suitable for mixing with food before oral administration once or twice daily to patients with difficulties swallowing.

57. The minitablet of claim 56, wherein the patients are between about 1 year to about 12 years of age and weighing between about 7 kg to less than about 40 kg.

58. The minitablet of claim 56, wherein the patients are elderly.

59. A pharmaceutical minitablet comprising about 0.1 mg to about 5 mg of mitapivat or a pharmaceutically acceptable salt thereof in an amount that is equivalent to about 0.1 mg to about 5 mg of mitapivat and having as its longest dimension or diameter a length of about 10.0 mm to about 0.1 mm for oral administration once or twice daily to patients that are between about 1 year to about 12 years of age and weighing between about 7 kg to less than about 40 kg or adult patients with difficulties swallowing.

60. The minitablet of claim 59, wherein the minitablet is suitable for mixing with food before administration.

61. The minitablet or granule of any one of claims 56 to 60, wherein the minitablet comprises about 1 mg of mitapivat or a pharmaceutically acceptable salt thereof in an amount that is equivalent to about 1 mg of mitapivat.

62. The minitablet or granule of any of claims 56 to 61 wherein the minitablet further comprises one or more of microcrystalline cellulose, mannitol, croscarmellose sodium and sodium stearyl fumarate.

63. A method of treating a condition selected from pyruvate kinase deficiency, sickle cell disease, and thalassemia in a pediatric subject about 1 year of age to about 12 years of age comprising administering to the pediatric subject an initial amount of mitapivat or a pharmaceutically acceptable salt thereof, once or twice daily, for a first period of time, wherein the initial amount administered is:
ii) about 0.5 mg to about 25 mg of mitapivat or an amount of a pharmaceutically salt of mitapivat that is equivalent to about 0.5 mg to about 25 mg of mitapivat if the pediatric subject's body weight is about 7 kg to less than about 40 kg;
iv) about 0.5 mg to about 25 mg of mitapivat or an amount of a pharmaceutically acceptable salt that is equivalent to about 0.5 mg to about 25 mg of mitapivat if the pediatric subject's body weight is about 7 kg to less than about 20 kg; or
v) about 0.5 mg to about 15 mg of mitapivat or an amount of a pharmaceutically acceptable salt that is equivalent to about 0.5 mg to about 15 mg of mitapivat if the pediatric subject is about 1 year of age to less than about 2 years of age and the pediatric subject's body weight is at least 7 kg;
assessing the body weight of the pediatric subject over a period of time; and
continuing to administer the initial amount of mitapivat or a pharmaceutically acceptable salt thereof if the pediatric subject's body weight remains within the same range, or administering a first adjusted amount of mitapivat or a pharmaceutically acceptable salt thereof based on the pediatric subject's weight falling within the ranges listed in i)-v) above.
